(19) United States
(12) Patent Application Publication
(10) Pub. No.: US 2008/0076810 A1
(43) Pub. Date: Mar. 27, 2008

(54) Benzoureas Having Anti-Diabetic Activity

(76) Inventors: Weiguo Lui, Princeton, NJ (US); Harold B. Wood, Westfield, NJ (US); Fiona Wai-Yu Lau-Phua, Edgewater, NJ (US)

Correspondence Address: MERCK AND CO., INC, P O BOX 2000, RAHWAY, NJ, 07065-0907, US

(21) Appl. No.: 11/597,817

(22) PCT Filed: May 26, 2005
(86) PCT No.: PCT/US05/18721

§ 371 Date: Nov. 21, 2006

Related U.S. Application Data
(60) Provisional application No. 60/575,144, filed on May 28, 2004.

(51) Int. Cl. A61K 31/422; A61K 31/428; A61P 3/00; C07D 413/14; C07D 417/14

(52) U.S. Cl. 514/367; 514/376; 548/159; 548/227

(57) ABSTRACT

Benzourea compounds of Formula I having aryl-(CH₂)ₓ-oxazolidinedione or aryl-(CH₂)ₓ-thiazolidinedione substituents on one of the N atoms of the benzourea ring, wherein x is 0 or 1, are PPAR gamma agonists or partial agonists and are useful in the treatment and control of type II diabetes, including hyperglycemia and other symptoms such as dyslipidemia, hyperlipidemia, hypercholesterolemia, hypertriglyceridemia, and obesity, that are often associated with type 2 diabetes.

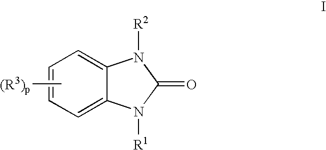

### FIELD OF THE INVENTION

The instant invention is concerned with benzoureas and pharmaceutically acceptable salts and prodrugs thereof, which are useful as therapeutic compounds, particularly in the treatment of Type 2 diabetes mellitus, and of conditions that are often associated with this disease, including obesity and lipid disorders.

### BACKGROUND OF THE INVENTION

Diabetes is a disease derived from multiple causative factors and characterized by elevated levels of plasma glucose (hyperglycemia) in the fasting state or after administration of glucose during an oral glucose tolerance test. There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin-dependent diabetes mellitus (NIDDM), insulin is still produced in the body. Patients having type 2 diabetes often have hyperinsulinemia (elevated plasma insulin levels); however, these patients are insulin resistant, which means that they have a resistance to the effect of insulin in stimulating glucose and lipid metabolism in the main insulin-sensitive tissues, which are muscle, liver and adipose tissues. Patients who are insulin resistant but not diabetic compensate for the insulin resistance by secreting more insulin, so that serum glucose levels are not elevated enough to meet the criteria of Type 2 diabetes. In patients with Type 2 diabetes, even elevated plasma insulin levels are insufficient to overcome the pronounced insulin resistance.

Persistent or uncontrolled hyperglycemia that occurs with diabetes is associated with increased and premature morbidity and mortality. Often abnormal glucose homeostasis is associated both directly and indirectly with obesity, hypertension, and alterations of the lipid, lipoprotein and apolipoprotein metabolism, as well as other metabolic and hemodynamic disease. Patients with type 2 diabetes mellitus have a significantly increased risk of macrovascular and microvascular complications, including atherosclerosis, coronary heart disease, stroke, peripheral vascular disease, hypertension, nephropathy, neuropathy, and retinopathy. Therefore, therapeutic control of glucose homeostasis, lipid metabolism, obesity, and hypertension are critically important in the clinical management and treatment of diabetes mellitus.

Many patients who have insulin resistance or Type 2 diabetes often have several symptoms that together are referred to as syndrome X, or the metabolic syndrome. A patient having this syndrome is characterized as having three or more symptoms selected from the following group of five symptoms: (1) abdominal obesity; (2) hypertriglyceridemia; (3) low high-density lipoprotein cholesterol (HDL); (4) high blood pressure; and (5) elevated fasting glucose, which may be in the range characteristic of Type 2 diabetes if the patient is also diabetic. Each of these symptoms is defined in the recently released Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670. Patients with metabolic syndrome, whether or not they have or develop overt diabetes mellitus, have an increased risk of developing the macrovascular and microvascular complications that are listed above that occur with type 2 diabetes, such as atherosclerosis and coronary heart disease.

Insulin resistance is not primarily caused by a diminished number of insulin receptors but by a post-insulin receptor binding defect that is not yet completely understood. This lack of responsiveness to insulin results in insufficient insulin-mediated activation of uptake, oxidation and storage of glucose in muscle and inadequate insulin-mediated repression of lipolysis in adipose tissue and of glucose production and secretion in the liver.

There are several available treatments for type 2 diabetes, each of which has its own limitations and potential risks. Physical exercise and a reduction in dietary intake of calories often dramatically improve the diabetic condition and are the best first line treatment of type 2 diabetes. Compliance with this treatment is very poor because of well-entrenched sedentary lifestyles and excess food consumption, especially of foods containing high amounts of fat. A widely used drug treatment involves the administration of meglitinide or a sulfonylurea (e.g. tolbutamide or glipizide), which are insulin secretagogues. These drugs increase the plasma level of insulin by stimulating the pancreatic β-cells to secrete more insulin. When administration of a sulfonylurea or meglitinide becomes ineffective, the amount of insulin in the body can be supplemented by the injection of insulin so that insulin concentrations are high enough to stimulate even the very insulin-resistant tissues. However, dangerously low levels of plasma glucose can result from administration of insulin and/or insulin secretagogues, and an increased level of insulin resistance due to the even higher plasma insulin levels can occur.

The biguanides are another class of drugs that are widely used to treat type 2 diabetes. The two best known biguanides, phenformin and metformin, cause some correction of hyperglycemia without risk of causing hypoglycemia. The biguanides can be used either with insulin or with an insulin secretagogue without increasing the risk of hypoglycemia. However, phenformin and metformin can induce lactic acidosis and nausea/diarrhea. Metformin has a lower risk of side effects than phenformin and is widely prescribed for the treatment of Type 2 diabetes.

The glitazones (i.e. 5-benzylthiazolidine-2,4-diones) are a newer class of compounds that can ameliorate hyperglycemia and other symptoms of type 2 diabetes. These agents substantially increase insulin sensitivity in muscle, liver and adipose tissue in several animal models of type 2 diabetes, resulting in partial or complete correction of elevated plasma glucose levels without the occurrence of hypoglycemia. The glitazones that are currently marketed (rosiglitazone and pioglitazone) are agonists of the peroxisome proliferator activated receptor (PPAR) gamma subtype. PPAR-gamma agonism is generally believed to be responsible for the improved insulin sensitization that is observed with the glitazones. New PPAR agonists are being developed for the treatment of Type 2 diabetes and/or dyslipidemia. Many of the newer PPAR compounds are agonists of one or more of the PPAR alpha, gamma and delta subtypes. Compounds that are agonists of both the PPAR alpha and PPAR gamma subtypes (PPAR alpha/gamma dual agonists) are promising because they reduce hyperglycemia and also improve lipid metabolism.

PPAR agonists, and particularly glitazones, have had shortcomings which have so far detracted from their attractiveness. Some of the compounds, especially troglitazone, have exhibited liver toxicity. Troglitazone was eventually withdrawn from the marketplace because of hepatotoxicity. Another weakness in the currently marketed PPAR agonists is that monotherapy for type 2 diabetes produces only modest efficacy—a reduction in average plasma glucose of ≈20% and a decline from ≈9.0% to ≈8.0% in HemoglobinA1C. The current compounds also do not greatly improve lipid metabolism, and may actually have a negative effect on the lipid profile. These shortcomings have provided an incentive to develop better insulin sensitizers for Type 2 diabetes which function via similar mechanism(s) of action.

Recently, there have been reports of compounds that are PPAR gamma antagonists or partial agonists. WO01/30343 describes a specific compound that is a PPAR partial agonist/antagonist that is useful for the treatment of obesity and Type 2 diabetes. WO02/08188, WO2004/020408, WO2004/020409, and WO2004/019869 disclose classes of PPAR agonists and partial agonists that are indole derivatives and that are useful in the treatment of Type 2 diabetes, with reduced side effects relating to body and heart weight gain. Benzoureas have not been disclosed as having anti-diabetic activity.

### SUMMARY OF THE INVENTION

The class of compounds described herein is a new class of PPAR-gamma agonists and partial agonists. The compounds are potent ligands of the PPAR gamma nuclear receptor. The class of compounds includes many compounds that are PPARγ partial agonists, but also may include PPARγ full agonists and/or PPARγ antagonists. Some compounds may also have PPARα activity in addition to PPARγ activity. The compounds are useful in the treatment and control of hyperglycemia and insulin resistance. The compounds are expected to be efficacious in the treatment of non-insulin dependent diabetes mellitus (NIDDM) in human and other mammalian patients, particularly in the treatment of hyperglycemia, and in the treatment of conditions associated with NIDDM, including hyperlipidemia, dyslipidemia, obesity, hypercholesterolemia, hypertriglyceridemia, atherosclerosis, vascular restenosis, inflammatory conditions, and other PPAR mediated diseases, disorders and conditions.

The compounds may also be useful in the treatment of one or more lipid disorders, including mixed or diabetic dyslipidemia, isolated hypercholesterolemia, which may be manifested by elevations in LDL-C and/or non-HDL-C, hyperapoBliproteinemia, hypertriglyceridemia, an increase in triglyceride-rich-lipoproteins, and low HDL cholesterol concentrations. They may also be useful in the treatment or amelioration of atherosclerosis, obesity, vascular restenosis, inflammatory conditions, psoriasis, polycystic ovary syndrome, and other PPAR mediated diseases, disorders and conditions.

The present invention is directed to compounds having formula I:

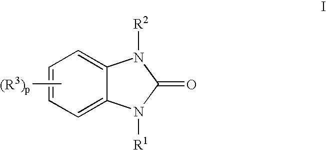

and pharmaceutically acceptable salts and prodrugs thereof, wherein
R¹ is —X-Aryl-Y-Z, and Aryl is optionally substituted with 1-3 groups independently selected from A;
Aryl is phenyl or naphthyl;
X and Y are each independently selected from a bond and —CR⁴R⁵—;
Z is

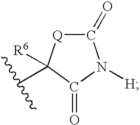

Q is selected from S and O;
A is selected from C₁-C₄ alkyl, C₂-C₄ alkenyl, —OC₁-C₄ alkyl, and halogen, wherein alkyl, alkenyl, and —Oalkyl are each optionally substituted with 1-5 halogens;
R² is selected from:
   (a) Benzisoxazolyl,
   (b) Aryl,
   (c) —(CH₂)Aryl,
   (d) —(C═O)Aryl, and
   (e) benzothiazolyl,
where R² is optionally substituted with 1-3 substituent groups which are independently selected from halogen, C₁-C₃ alkyl, and —OC₁-C₃ alkyl, wherein C₁-C₃ alkyl and —OC₁-C₃ alkyl are optionally substituted with 1-5 halogens;
R³, R⁴, and R⁵ are each independently selected from hydrogen, halogen, C₁-C₃ alkyl, and —OC₁-C₃ alkyl, where C₁-C₃ alkyl and —OC₁-C₃ alkyl are optionally substituted with 1-5 halogens;
R⁶ is selected from H, C₁-C₃ alkyl, and halogen, where C₁-C₃ alkyl is optionally substituted with 1-3F; and
p is an integer from 0 to 4.

In the above definitions and subsequent definitions, alkyl groups may be either linear or branched, unless otherwise specified.

### DETAILED DESCRIPTION OF THE INVENTION

The invention has numerous embodiments, as set forth below. All embodiments also include pharmaceutically acceptable salts of the compounds that are defined.

One embodiment comprises compounds having Formula I, wherein R¹ is —X-phenyl-YZ.

In other embodiments of the compounds of Formula I, X and Y each independently can be a bond or —CH₂—.

In other embodiments of the compounds of Formula I, Aryl is optionally substituted with 1-2 groups A, where each group A can be halogen, —CF₃, —OCF₃, —CH₃, or —OCH₃.

In other embodiments of the compounds of Formula I, R³ is —CH₃, —OCH₃, —OCF₃, or —CF₃.

In other embodiments, R⁶ is H, CH₃, or CF₃.

In other embodiments, p is 0 or 1.

In other embodiments of the compounds of Formula I, R² is 3-benzisoxazolyl, which is optionally substituted with 1-2 groups independently selected from halogen, —OCH₃, —OCF₃, CH₃, and CF_{3.}

In other embodiments of the compounds of Formula I, R² is 2-benzothiazolyl, which is optionally substituted with 1-2 groups independently selected from halogen, —OCH₃, —OCF₃, CH₃, and CF_{3.}

In other embodiments of the compounds of Formula I,
R¹ is —X-phenyl-YZ, where phenyl is optionally substituted with 1-2 groups independently selected from A;
X and Y are each independently selected from a bond and —CH₂—;
A is selected from halogen, —CF₃, —OCF₃, —CH₃, and —OCH₃;
R³ is selected from —CF₃, —OCF₃, —CH₃, and —OCH₃;
R⁶ is selected from H, —CH₃, and —CF₃; and
p is 0 or 1.

A subset of the compounds of Formula I, or of any of the embodiments described above, includes compounds in which Q is O, X is a bond, and Y is —CH₂—.

Another subset of the compounds of Formula I, or of any of the embodiments described above, includes compounds in which Q is O, X is —CH₂—, and Y is a bond.

Another subset of the compounds of Formula I, or of any of the embodiments described above, includes compounds in which Q is O and X and Y are each —CH₂—.

Another subset of the compounds of Formula I, or of any of the embodiments described above, includes compounds in which Q is O and X and Y are each a bond.

Other subsets of the compounds of Formula I, or of any of the embodiments described above, include compounds in which Q is S.

The invention includes compounds of Formula I, including pharmaceutically acceptable salts of these compounds, prodrugs of these compounds, and pharmaceutical compositions comprising these compounds and a pharmaceutically acceptable carrier.

Structures of specific compounds are disclosed in the examples and in Table 1. The syntheses of specific compounds are also provided hereinafter in the Examples.

The compounds of this invention can be used in pharmaceutical compositions comprising the compound or a pharmaceutically acceptable salt thereof and a pharmaceutically acceptable carrier. The compounds of this invention can also be used in pharmaceutical compositions in which a compound of Formula I or a pharmaceutically acceptable salt thereof is the only active ingredient.

The compounds of the invention and pharmaceutically acceptable salts thereof can be used in the manufacture of medicaments for the treatment of type 2 diabetes mellitus in a human or other mammalian patient, and in the manufacture of medicaments for other diseases described herein that are treated by the compounds.

The compounds as defined above may be used in any of the following methods to treat or control diseases, as well as methods to treat other diseases not listed below, in a mammalian patient, especially a human, by administering to the patient a therapeutically effective amount of a compound of Formula I:
(1) non-insulin dependent diabetes mellitus (type 2 diabetes);
(2) hyperglycemia;
(3) metabolic syndrome;
(4) obesity;
(5) hypercholesterolemia;
(6) hypertriglyceridemia; and/or
(7) one or more lipid disorders, including mixed or diabetic dyslipidemia, low HDL cholesterol, high LDL cholesterol, hyperlipidemia, hypercholesterolemia, and hypertriglyceridemia.

The compounds may also be used in a method for reducing the risks of adverse sequelae associated with metabolic syndrome in a human or other mammalian patient in need of such treatment which comprises administering to the patient a therapeutically effective amount of a compound of Formula I.

The compounds may also be used in a method for treating atherosclerosis, for reducing the risk of developing atherosclerosis, for delaying the onset of atherosclerosis, and/or reducing the risk of sequelae of atherosclerosis in a human or other mammalian patient in need of such treatment or at risk of developing atherosclerosis or sequelae of atherosclerosis, which comprises administering to the patient a therapeutically effective amount of a compound of Formula I. Sequelae of atherosclerosis include for example angina, claudication, heart attack, stroke, etc.

The compounds are especially useful in the treatment of the following diseases, by administering a therapeutically effective amount to a patient in need of treatment:
(1) type 2 diabetes, and especially hyperglycemia resulting from type 2 diabetes;
(2) metabolic syndrome;
(3) obesity; and
(4) hypercholesterolemia.

### Definitions

“Ac” is acetyl, which is CH₃C(O)—.

“Alkyl” means saturated carbon chains which may be linear or branched or combinations thereof, unless the carbon chain is defined otherwise. Other groups having the prefix “alk”, such as alkoxy and alkanoyl, also may be linear or branched or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic saturated or partially unsaturated carbocyclic rings, each having from 3 to 10 carbon atoms, unless otherwise stated. The term also includes a monocyclic ring fused to an aryl group. Examples of cycloalkyl include cyclopropyl, cyclopentyl, cyclohexyl, cycloheptyl, and the like.

A cycloalkylidene group is a divalent cycloalkane radical in which both attachments are at the same carbon. For example, the cyclopropyl group of 1,1-dimethylcyclopropane is a cyclopropylidene group.

“Aryl” (and “arylene”) when used to describe a substituent or group in a structure means a monocyclic or bicyclic compound in which all the rings are aromatic and which contains only carbon ring atoms. The term “aryl” can also refer to an aryl group that is fused to a cycloalkyl or heterocycle. “Heterocyclyl,” “heterocycle,” and “heterocyclic” means a fully or partially saturated monocyclic or bicyclic ring system containing 1-4 heteroatoms independently selected from N, S and O, each of said rings having from 3 to 8 atoms. Examples of aryl substituents include phenyl and naphthyl. Aryl rings fused to cycloalkyls are found in indanyl, indenyl, and tetrahydronaphthyl. Examples of aryl fused to heterocyclic groups are found in 2,3-dihydrobenzofuranyl, dihydrobenzopyranyl, and the like. Examples of heterocycles include tetrahydrofuran, piperazine, and morpholine. Preferred aryl groups are phenyl and naphthyl. Phenyl is generally the most preferred.

“Heteroaryl” (and heteroarylene) means a mono- or bicyclic aromatic ring system containing 1-4 heteroatoms selected from N, O and S, including —S(O)— and —S(O)₂—, with each ring containing 5 to 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzisoxazolyl, benzoxazolyl, benzothiazolyl, benzimidazolyl, benzofuranyl, benzothiophenyl (including S-oxide and dioxide), furo(2,3-b)pyridyl, quinolyl, indolyl, isoquinolyl, and the like. Preferred heteroaryl groups include pyridyl (2-, 3-, and 4-pyridyl) and quinolyl.

“Halogen” includes fluorine, chlorine, bromine and iodine.

“Me” represents methyl.

The term “composition,” as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The substituent “tetrazole” means a 2H-tetrazol-5-yl substituent group and tautomers thereof.

### Optical Isomers—Diastereomers—Geometric Isomers—Tautomers

Compounds of Formula I may contain one or more asymmetric centers and can thus occur as racemates, racemic mixtures, single enantiomers, diastereomeric mixtures and individual diastereomers. The present invention is meant to comprehend all such isomeric forms of the compounds of Formula I.

Some of the compounds described herein may contain olefinic double bonds, and unless specified otherwise, are meant to include both E and Z geometric isomers.

Some of the compounds described herein may exist with different points of attachment of hydrogen, referred to as tautomers. An example is a ketone and its enol form, known as keto-enol tautomers. The individual tautomers as well as mixtures thereof are encompassed with compounds of Formula I.

Compounds of Formula I having one or more asymmetric centers may be separated into diastereoisomers, enantiomers, and the like by methods well known in the art.

Alternatively, enantiomers and other compounds with chiral centers may be synthesized by stereospecific synthesis using optically pure starting materials and/or reagents of known configuration.

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

### Utilities

Compounds of the present invention are potent ligands having agonist, partial agonist or antagonist activity on one or more of the various peroxisome proliferator activated receptor subtypes, particularly PPARγ. The compounds may also be ligands or agonists, partial agonists or antagonists of the PPARα subtype as well as the PPARγ subtype, resulting in mixed PPARα/γ agonism. Some compounds (generally less preferred) may also be PPARδ ligands and have PPARδ activity in addition to their other PPAR activity. The compounds of this invention are useful in treating or controlling diseases, disorders or conditions which are mediated by one or more ligands of the individual PPAR subtypes (eg. γ or α) or a combination of PPAR subtypes (e.g. α/γ). One aspect of the present invention provides a method for the treatment and control of diseases that can be mediated by administration of a PPAR agonist or partial agonist, particularly a PPARγ agonist or partial agonist, such as type 2 diabetes. One aspect of the present invention provides a method for the treatment and control of such diseases, disorders, or conditions in a mammal which comprises administering to such mammal a therapeutically effective amount of a compound of Formula I. Compounds of the present invention may be useful in treating or controlling many PPAR mediated diseases and conditions, including, but not limited to, (1) diabetes mellitus, and especially non-insulin dependent diabetes mellitus (NIDDM), (2) hyperglycemia, (3) low glucose tolerance, (4) insulin resistance, (5) obesity, (6) lipid disorders, (7) dyslipidemia, (8) hyperlipidemia, (9) hypertriglyceridemia, (10) hypercholesterolemia, (11) low HDL levels, (12) high LDL levels, (13) atherosclerosis and its sequelae, (14) vascular restenosis, (15) irritable bowel syndrome, (16) inflammatory bowel disease, including Crohn's disease and ulcerative colitis, (17) other inflammatory conditions, (18) pancreatitis, (19) abdominal obesity, (20) neurodegenerative disease, (21) retinopathy, (22) psoriasis, (23) metabolic syndrome, (24) ovarian hyperandrogenism (polycystic ovarian syndrome), and other disorders where insulin resistance is a component. They may also have utility in treating high blood pressure, neoplastic conditions, adipose cell tumors, adipose cell carcinomas, such as liposarcoma, prostate cancer and other cancers, including gastric, breast, bladder and colon cancers, angiogenesis, and Alzheimer's disease.

The present compounds can be used to lower glucose, lipids, and insulin in non-diabetic patients that have impaired glucose tolerance and/or are in a pre-diabetic condition.

The present compounds can be used to treat obesity in a patient in need of such treatment by administering to the patient a therapeutically effective amount of a compound of Formula I.

The present compounds can be used to treat or reduce the risk of developing atherosclerosis in a patient in need of such treatment by administering to the patient a therapeutically effective amount of a compound of Formula 1.

The present compounds can be used to treat or reduce hyperglycemia in a diabetic patient in need of such treatment by administering to the patient a therapeutically effective amount of a compound of Formula 1.

The compounds may have utility in treating osteoporosis. The compounds of this invention may treat osteoporosis or reduce the risk of developing osteoporosis by slowing or stopping the loss of bone density in a patient who has osteoporosis or is at risk of developing osteoporosis. The compounds of this invention may also reverse the loss of bone mass in patients who have already begun to lose bone mass.

One aspect of the invention provides a method for the treatment and control of mixed or diabetic dyslipidemia, hypercholesterolemia, atherosclerosis, low HDL levels, high LDL levels, hyperlipidemia, and/or hypertriglyceridemia, which comprises administering to a patient in need of such treatment a therapeutically effective amount of a compound having formula I. The compound may be used alone or advantageously may be administered with a cholesterol biosynthesis inhibitor, particularly an HMG-CoA reductase inhibitor such as lovastatin, simvastatin, rosuvastatin, pravastatin, fluvastatin, atorvastatin, rivastatin, itavastatin, or ZD4522. The compound may also be used advantageously in combination with other lipid lowering drugs such as cholesterol absorption inhibitors (for example stanol esters, sterol glycosides such as tiqueside, and azetidinones such as ezetimibe), ACAT inhibitors (such as avasimibe), CETP inhibitors, niacin, niacin receptor agonists, bile acid sequestrants, microsomal triglyceride transport inhibitors, and bile acid reuptake inhibitors. These combination treatments may also be effective for the treatment or control of one or more related conditions selected from the group consisting of hypercholesterolemia, atherosclerosis, hyperlipidemia, hypertriglyceridemia, dyslipidemia, high LDL, and low HDL.

Another aspect of the invention provides a method of treating inflammatory conditions, including inflammatory bowel disease, Crohn's disease, and ulcerative colitis by administering an effective amount of a compound of this invention to a patient in need of treatment. Additional inflammatory diseases that may be treated with the instant invention include gout, rheumatoid arthritis, osteoarthritis, multiple sclerosis, asthma, ARDS, psoriasis, vasculitis, ischemia/reperfusion injury, frostbite, and related diseases.

### Administration and Dose Ranges

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of Formula I are administered orally.

The effective dosage of active ingredient employed may vary depending on the particular compound employed, the mode of administration, the condition being treated and the severity of the condition being treated. Such dosage may be ascertained readily by a person skilled in the art.

When treating or controlling diabetes mellitus and/or hyperglycemia or hypertriglyceridemia or other diseases for which compounds of Formula I are indicated, generally satisfactory results are obtained when the compounds of the present invention are administered at a daily dosage of from about 0.01 milligram to about 100 milligrams per kilogram of animal body weight, preferably given as a single daily dose or in divided doses two to six times a day, or in sustained release form. For most large mammals, including humans (e.g. a 70 kg adult), the total daily dosage is from about 0.1 milligrams to about 1000 milligrams, is likely to be from about 0.5 milligrams to about 350 milligrams, and is often from about 1 milligram to about 50 milligrams. For a particularly potent compound, the dosage for an adult human may be as low as 0.1 mg. Examples of daily dosages for a 70 kg adult human are 0.1 mg, 0.5 mg, 1 mg, 2 mg, 5 mg, 10 mg, 25 mg, 50 mg, 100 mg, 150 mg, 200 mg, 250 mg, 350 mg, and 500 mg per day. The daily dosage regimen may be adjusted within the above ranges or even outside of these ranges to provide the optimal therapeutic response.

Oral administration will usually be carried out using tablets. Examples of doses in tablets which may be administered once a day or more than once a day (e.g. 2×, 3×, or (rarely) 4 or more times per day, are 0.1 mg, 0.5 mg, 1 mg, 2 mg, 5 mg, 10 mg, 25 mg, 50 mg, 100 mg, 150 mg, 200 mg, 250 mg, 350 mg, and 500 mg. Other oral forms (e.g. capsules or suspensions) can also be administered in doses having similar sizes.

### Pharmaceutical Compositions

Another aspect of the present invention provides pharmaceutical compositions which comprise a compound of Formula I and a pharmaceutically acceptable carrier. The pharmaceutical compositions of the present invention comprise a compound of Formula I or a pharmaceutically acceptable salt as an active ingredient, as well as a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids. A pharmaceutical composition may also comprise a prodrug, or a pharmaceutically acceptable salt thereof, if a prodrug is administered.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy. In general, compositions suitable for oral administration are preferred.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form, in which case solid pharmaceutical carriers are employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

### Combination Therapy

Compounds of Formula I may be used in combination with other drugs that may also be useful in the treatment or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy also includes therapies in which the compound of Formula I and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of Formula I, and either administered separately or in the same pharmaceutical composition, include, but are not limited to:
(a) other PPAR gamma agonists and partial agonists, such as the glitazones (e.g. troglitazone, pioglitazone, englitazone, MCC-555, rosiglitazone, balaglitazone, netoglitazone, and the like), and PPAR gamma agonists and partial agonists that do not have a glitazone structure;
(b) biguanides such as metformin and phenformin;
(c) protein tyrosine phosphatase-1B (PTP-1B) inhibitors,
(d) dipeptidyl peptidase IV (DP-IV) inhibitors, such as MK-0431, LAF-237, BMS477118, PSN-9301, and GSK-823093;
(e) insulin or insulin mimetics;
(f) sulfonylureas such as tolbutamide and glipizide, or related materials;
(g) α-glucosidase inhibitors (such as acarbose);
(h) agents which improve a patient's lipid profile, such as (i) HMG-CoA reductase inhibitors (lovastatin, simvastatin, rosuvastatin, pravastatin, fluvastatin, atorvastatin, rivastatin, itavastatin, ZD-4522 and other statins), (ii) bile acid sequestrants (cholestyramine, colestipol, and dialkylaminoalkyl derivatives of a cross-linked dextran), (iii) nicotinyl alcohol, nicotinic acid or a salt thereof, (iv) niacin receptor agonists, (v) PPARα agonists such as fenofibric acid derivatives (gemfibrozil, clofibrate, fenofibrate and bezafibrate), (vi) cholesterol absorption inhibitors, such as for example ezetimibe, (vii) acyl CoA:cholesterol acyltransferase (ACAT) inhibitors, such as avasimibe, (viii) CETP inhibitors, and (ix) phenolic anti-oxidants, such as probucol;
(i) PPARα/γ dual agonists, such as KRP-297, muraglitazar, tesaglitazar, LY-818 and the like;
(j) PPARδ agonists such as those disclosed in WO97/28149;
(k) antiobesity compounds such as fenfluramine, dexfenfluramine, phentiramine, subitramine, orlistat, neuropeptide Y5 inhibitors, Mc4r agonists, cannabinoid receptor 1 (CB-1) antagonists/inverse agonists, and β₃ adrenergic receptor agonists;
(l) ileal bile acid transporter inhibitors;
(m) agents intended for use in inflammatory conditions such as aspirin, non-steroidal anti-inflammatory drugs, glucocorticoids, azulfidine, and cyclo-oxygenase 2 selective inhibitors;
(n) glucagon receptor antagonists;
(o) GLP-1,
(p) GIP-1, and
(q) GLP-1 analogs, such as exendins.

The above combinations include combinations of a compound of the present invention not only with one other active compound, but also with two or more other active compounds. Non-limiting examples include combinations of compounds having Formula I with two or more active compounds selected from biguanides, sulfonylureas, HMG-CoA reductase inhibitors, other PPAR agonists, PTP-1B inhibitors, DP-IV inhibitors, and anti-obesity compounds.

Compounds of the present invention (i.e. compounds having Formula I) can be used to treat one or more diseases or conditions selected from hypercholesterolemia, atherosclerosis, low HDL levels, high LDL levels, hyperlipidemia, hypertriglyceridemia, and dyslipidemia by administering a therapeutically effective amount of a compound of Claim 1 in combination with an HMG-CoA reductase inhibitor to a patient in need of such treatment. Statins are the preferred HMG-CoA reductase inhibitors for use in this combination therapy. Preferred statins include lovastatin, simvastatin, pravastatin, fluvastatin, atorvastatin, itavastatin, ZD-4522, rivastatin, and rosuvastatin. This combination treatment may be particularly desirable for treating or reducing the risk of developing atherosclerosis.

### Biological Assays

### A) PPAR Binding Assays

For preparation of recombinant human PPARγ, PPARδ, and PPARα: Human PPARγ₂, human PPARδ and human PPARα were expressed as gst-fusion proteins in E. coli. The full length human cDNA for PPARγ₂ was subcloned into the pGEX-2T expression vector (Pharmacia). The full length human cDNAs for PPARδ and PPARα were subcloned into the pGEX-KT expression vector (Pharmacia). E. coli containing the respective plasmids were propagated, induced, and harvested by centrifugation. The resuspended pellet was broken in a French press and debris was removed by centrifugation at 12,000×g. Recombinant human PPAR receptors were purified by affinity chromatography on glutathione sepharose. After application to the column, and one wash, receptor was eluted with glutathione. Glycerol (10%) was added to stabilize the receptor and aliquots were stored at −80° C.

For binding to PPARγ, an aliquot of receptor was incubated in TEGM (10 mM Tris, pH 7.2, 1 mM EDTA, 10% glycerol, 7 μL/100 mL β-mercaptoethanol, 10 mM Na molybdate, 1 mM dithiothreitol, 5 μg/mL aprotinin, 2 μg/mL leupeptin, 2 μg/mL benzamidine and 0.5 mM PMSF) containing 0.1% non-fat dry milk and 10 nM [³H₂] AD5075, (21 Ci/mmole), ±test compound as described in Berger et al (Novel peroxisome proliferator-activated receptor (PPARγ) and PPARδ ligands produce distinct biological effects. J. Biol. Chem. (1999), 274: 6718-6725. Assays were incubated for ˜16 hr at 4° C. in a final volume of 150 μL. Unbound ligand was removed by incubation with 100 μL dextran/gelatin-coated charcoal, on ice, for ˜10 min. After centrifugation at 3000 rpm for 10 min at 4° C., 50 μL of the supernatant fraction was counted in a Topcount.

For binding to PPARδ, an aliquot of receptor was incubated in TEGM (10 mM Tris, pH 7.2, 1 mM EDTA, 10% glycerol, 7 μL/100 mL β-mercaptoethanol, 10 mM Na molybdate, 1 mM dithiothreitol, 5 μg/mL aprotinin, 2 μg/mL leupeptin, 2 μg/mL benzamide and 0.5 mM PMSF) containing 0.1% non-fat dry milk and 2.5 nM [³H₂]L-783483, (17 Ci/mmole), ±test compound as described in Berger et al (Novel peroxisome proliferator-activated receptorγ (PPARγ) and PPARδ ligands produce distinct biological effects. 1999 J Biol Chem 274: 6718-6725). (L-783483 is 3-chloro-4-(3-(7-propyl-3-trifluoromethyl-6-benz-[4,5]-isoxazoloxy)propylthio)phenylacetic acid, Ex. 20 in WO 97/28137). Assays were incubated for ˜16 hr at 4° C. in a final volume of 150 μL. Unbound ligand was removed by incubation with 100 μL dextran/gelatin-coated charcoal, on ice, for ˜10 min. After centrifugation at 3000 rpm for 10 min at 4° C., 50 μL of the supernatant fraction was counted in a Topcount.

For binding to PPARα, an aliquot of receptor was incubated in TEGM (10 mM Tris, pH 7.2, 1 mM EDTA, 10% glycerol, 7 μL/100 mL β-mercaptoethanol, 10 mM Na molybdate, 1 mM dithiothreitol, 5 μg/mL aprotinin, 2 μg/mL leupeptin, 2 μg/mL benzamide and 0.5 mM PMSF) containing 0.1% non-fat dry milk and 5.0 nM [³H₂]L-797773, (34 Ci/mmole), ±test compound. (L-797733 is (3-(4-(3-phenyl-7-propyl-6-benz-[4,5]-isoxazoloxy)butyloxy))phenylacetic acid, Ex. 62 in WO 97/28137). Assays were incubated for ˜16 hr at 4° C. in a final volume of 150 μL. Unbound ligand was removed by incubation with 100 μL dextran/gelatin-coated charcoal, on ice, for ˜10 min. After centrifugation at 3000 rpm for 10 min at 4° C., 50 μL of the supernatant fraction was counted in a Topcount.

### B) Gal-4hPPAR Transactivation Assays

The chimeric receptor expression constructs, pcDNA3-hPPARγ/GAL4, pcDNA3-hPPARδ/GAL4, pcDNA3-hPPARα/GAL4 were prepared by inserting the yeast GAL4 transcription factor DBD adjacent to the ligand binding domains (LBDs) of hPPARγ, hPPARδ, hPPARα, respectively. The reporter construct, pUAS(5×)-tk-luc was generated by inserting 5 copies of the GAL4 response element upstream of the herpes virus minimal thymidine kinase promoter and the luciferase reporter gene. pCMV-lacZ contains the galactosidase Z gene under the regulation of the cytomegalovirus promoter. COS-1 cells were seeded at 12×10³ cells/well in 96 well cell culture plates in high glucose Dulbecco's modified Eagle medium (DMEM) containing 10% charcoal stripped fetal calf serum (Gemini Bio-Products, Calabasas, Calif.), nonessential amino acids, 100 units/ml Penicillin G and 100 mg/ml Streptomycin sulfate at 37° C. in a humidified atmosphere of 10% CO₂. After 24 h, transfections were performed with Lipofectamine (GIBCO BRL, Gaithersburg, Md.) according to the instructions of the manufacturer. Briefly, transfection mixes for each well contained 0.48 μl of Lipofectamine, 0.00075 μg of pcDNA3-PPAR/GAL4 expression vector, 0.045 μg of pUAS(5×)-tk-luc reporter vector and 0.0002 μg of pCMV-lacZ as an internal control for transactivation efficiency. Cells were incubated in the transfection mixture for 5 h at 37° C. in an atmosphere of 10% CO₂. The cells were then incubated for ˜48 h in fresh high glucose DMEM containing 5% charcoal stripped fetal calf serum, nonessential amino acids, 100 units/mil Penicillin G and 100 mg/ml Streptomycin sulfate±increasing concentrations of test compound. Since the compounds were solubilized in DMSO, control cells were incubated with equivalent concentrations of DMSO; final DMSO concentrations were ≦0.1%, a concentration which was shown not to effect transactivation activity. Cell lysates were produced using Reporter Lysis Buffer (Promega, Madison, Wis.) according to the manufacturer's instructions. Luciferase activity in cell extracts was determined using Luciferase Assay Buffer (Promega, Madison, Wis.) in an ML3000 luminometer (Dynatech Laboratories, Chantilly, Va.). β-galactosidase activity was determined using β-D-galactopyranoside (Calbiochem, San Diego, Calif.).

Agonism is determined by comparison of maximal transactivation activity with a full PPAR agonist, such as rosiglitazone. Generally, if the maximal stimulation of transactivation is less than 50% of the effect observed with a full agonist, then the compound is designated as a partial agonist. If the maximal stimulation of transactivation is greater than 50% of the effect observed with a full agonist, then the compound is designated as a full agonist. The compounds of this invention generally have EC50 values in the range of 1 nM to 3000 nM.

### C) In Vivo Studies

Male db/db mice (10-11 week old C57B1/KFJ, Jackson Labs, Bar Harbor, Me.) are housed 5/cage and allowed ad lib. access to ground Purina rodent chow and water. The animals, and their food, are weighed every 2 days and are dosed daily by gavage with vehicle (0.5% carboxymethylcellulose)±test compound at the indicated dose. Drug suspensions are prepared daily. Plasma glucose, and triglyceride concentrations are determined from blood obtained by tail bleeds at 3-5 day intervals during the study period. Glucose and triglyceride, determinations are performed on a Boehringer Mannheim Hitachi 911 automatic analyzer (Boehringer Mannheim, Indianapolis, Ind.) using heparinized plasma diluted 1:6 (v/v) with normal saline. Lean animals are age-matched heterozygous mice maintained in the same manner.

### EXAMPLES

The following Schemes and Examples are provided to illustrate the invention and are not to be construed as limiting the invention in any manner. The scope of the invention is defined by the appended claims.

The structures in Table 1 further illustrate compounds of this invention that were made or can be made using the methods disclosed herein.

Compounds in Table 1 that were synthesized were analyzed using tandem high pressure liquid chromatography-mass spectrometry (LC-MS) and/or proton NMR. LC-MS samples were analyzed using an Agilent 1100 Series high pressure liquid chromatograph coupled to a Waters Micromass ZQ mass spectrometer. The column used was a Waters XTerra and compounds were eluted using a gradient elution program (10% B to 100% B in 4.5 min) with a flow rate of 2.5 mL/min; Solvent A: water containing 0.06% trifluoroacetic acid; Solvent B: acetonitrile containing 0.05% trifluoroacetic acid. Retention times are given in minutes.
Method A: XTerra MS-C18, 4.5×50 mm, 10-100% B in 4.5 min, flow rate 2.5 ml/min.
Method B: XTerra C18, 3×50 mm, 10-98% in 3.75 min, then 98% for 1 min, flow rate 1 ml/min.

General and specific procedures for making the compounds of this invention and synthetic intermediates are summarized below. Other compounds claimed herein can readily be made by a practitioner of medicinal and/or synthetic organic chemistry by adapting the procedures disclosed herein to the specific compound.

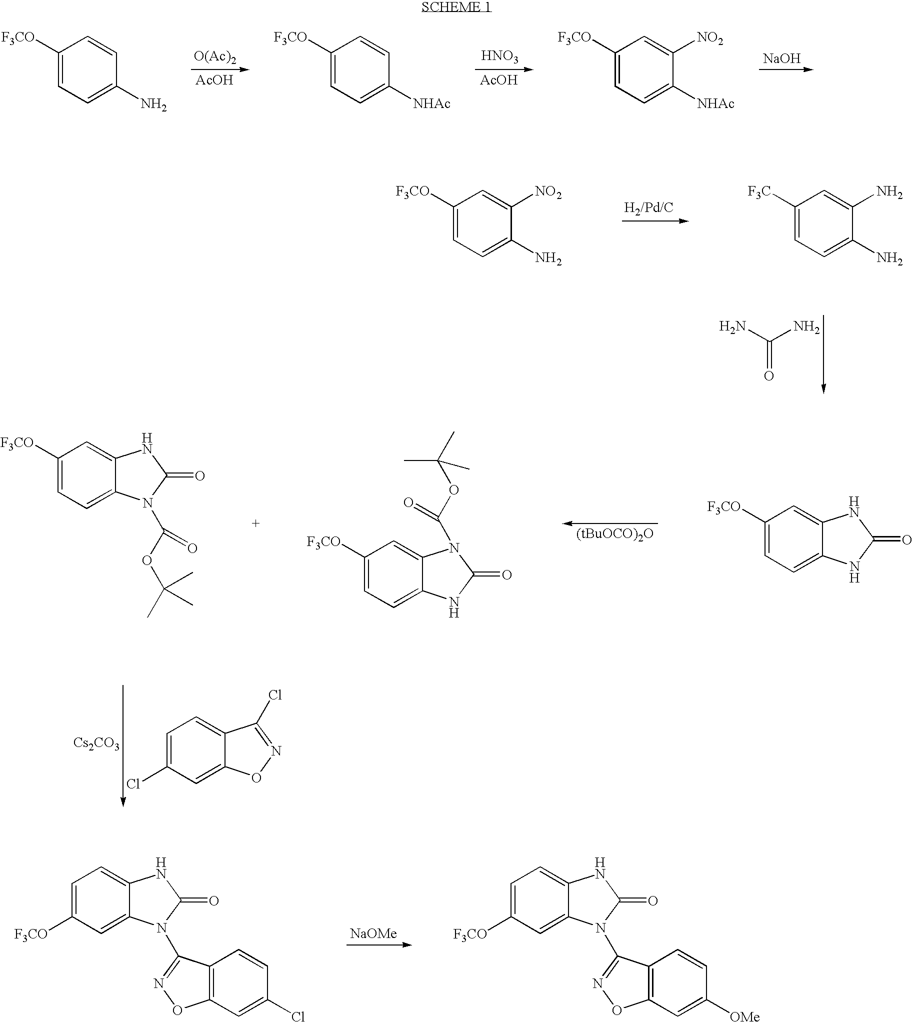

### Experimental Procedure:

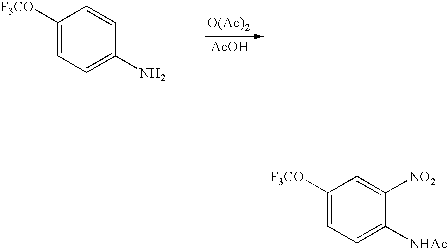

### 2-Nitro-4-trifluoromethoxyacetylaniline

To a solution of 4-trifluoromethoxyaniline (35 g, 200 mmol) in acetic acid (35 mL) at 0 to 10° C., was added acetic anhydride (75 mL). The reaction mixture was stirred at 10 to 30° C. for 30 min. While cooling in ice bath, H₂SO₄ (96%, 1.5 mL, 26 mmol) was added followed by HNO₃ (90%, 9.4 mL, 200 mmol). The mixture was stirred at room temperature for one hour and diluted with water (300 ml). The resulting slurry was stirred in ice bath for 30 minutes and filtered. The solid was washed with water and air dried to obtain a yellow solid. LC-MS ¹H NMR (CDCl₃, 500 MHz)

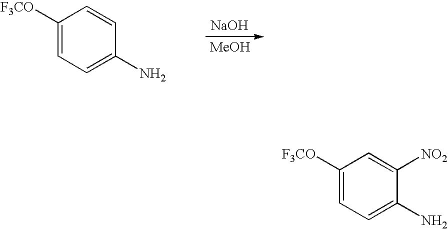

### 2-Nitro-4-trifluoromethoxyaniline

To a solution of 2-nitro-4-trifluoromethoxyacetylaniline (45 g, 170 mmol) in methanol (200 mL) at 0 to 10° C., was added sodium hydroxide solution (NaOH, 8.5 g, 220 mmol, in 45 mL water). The reaction mixture was stirred at room temperature for one hour and diluted with 300 mL of water. The slurry was stirred in an ice bath for two hours and filtered. The yellow solid was washed with 50 mL methanol:water (1:2) and dried under vacuum to obtain a yellow solid.

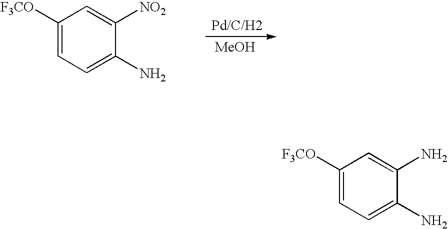

### 1,2-Diamino-4-trifluoromethoxybenzene

To a solution of 2-Nitro-4-trifluoromethoxyaniline (40 g, 180 mmol) in methanol (250 ml), was added 10% palladium on carbon (Pd/C, 10% wt/wt, 500 mg). The suspension was shaken in a Parr bottle under 45 psi of hydrogen for 4 hours, and then filtered through celite. The filtrate was concentrated to dryness to obtain a light gray solid.

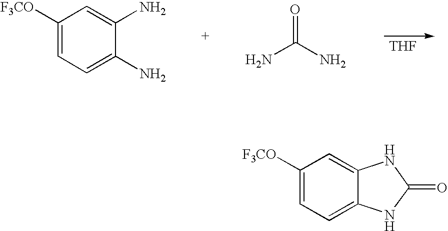

### 5-Trifluoromethoxybenzimidazole-2-one

To a solution of 1,2-diamino-4-trifluoromethoxybenzene (35 g, 180 mmol) in tetrahydrofuran (350 ml) was added urea (32 g, 180 mmol). The solution was refluxed at 70° C. overnight, concentrated under vacuum to a volume of about 100 mL and diluted with water (500 mL). The suspension was stirred at room temperature overnight and filtered. The solid was washed with water and dried under vacuum to obtain a white crystalline solid. LC-MS: m/e (M+1)=219. ¹H NMR (DMSO, 500 MHz) δ 10.85 (d, J=8 Hz, 2H), 6.97 (d, J=8.5 Hz, 1H), 6.90 (d, J=11 Hz, 2H).

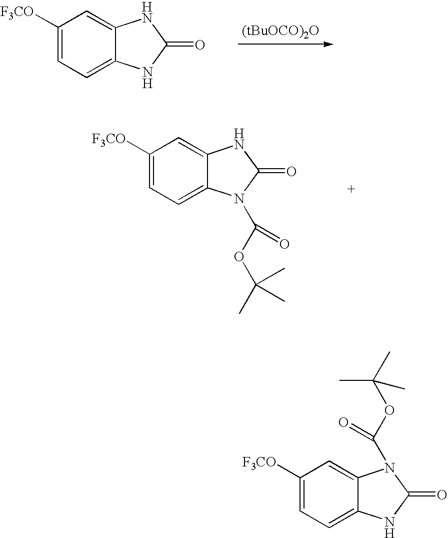

### 1-Boc-5-trifluoromethoxybenzimidazole-2-one and 1-Boc-6-trifluoromethoxybenzimidazole-2-one

To a solution of 5-trifluoromethoxybenzimidazole-2-one (35 g, 160 mmol) in DMF (150 mL) was added NaH (95%, 4.9 g, 190 mmol). The reaction mixture was stirred at room temperature for 2 hours followed by addition of di-tert-butyl dicarbonate (35 g, 160 mmol). The mixture was stirred at room temperature overnight, diluted with water (400 ml), and extracted with ethyl acetate (2×150 ml). The organic extract was washed with water (2×100 ml) and brine (100 ml), dried over anhydrous MgSO₄, and concentrated under vacuum. The residue was chromatographed on silica gel with hexane/ethyl acetate (3:1) as the solvent system to obtain 1-Boc-5-trifluoromethoxybenzimidazole-2-one as an earlier eluted product. Fractions containing the later eluted product were combined and concentrated to dryness to obtain 1-Boc-5-trifluoromethoxybenzimidazole-2-one. LC-MS (M+1)=319. ¹H NMR (DMSO, 500 MHz) δ 11.21 (brs, 1H), 7.56 (s, 1H), 7.12 (d, J=7 Hz, 1H), 7.03 (d, J=7 Hz, 1H),) 1.57 (s, 9H).

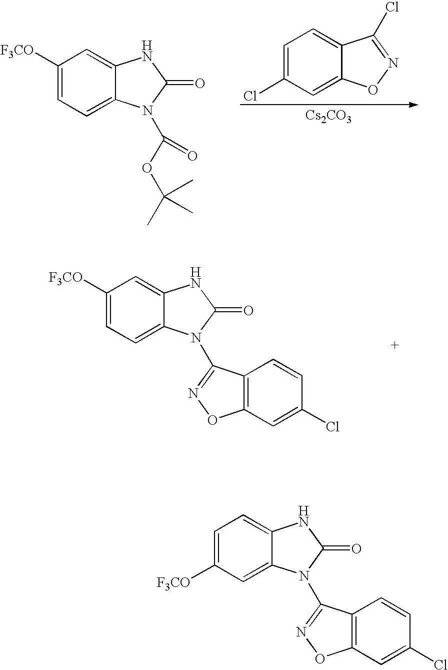

### 1-[(6-chloro)-benzisoxazol-3-yl]-5-trifluoromethoxylbenzimidazole-2-one and 1-[(6-chloro)-benzisoxazol-3-yl]-6-trifluoromethoxylbenzimidazole-2-one

To a solution of 1-Boc-5-trifluoromethoxybenzimidazole-2-one (5 g, 15.7 mmol) in DMF (20 ml) was added 3,6-dichlorobenzoisoxazole (3.0 g, 15.7 mmol) and Cs₂CO₃ (11 g, 31.4 mmol). The suspension was heated to 150° C. in an oil bath and stirred overnight. The mixture was then cooled to room temperature, diluted with water (30 ml) and extracted with ethyl acetate (2×20 ml). The organic extracts were combined, dried over anhydrous Mg₂SO₄, and concentrated to dryness. The residue was purified by silica gel column chromatography using hexane/ethyl acetate (4:1) as solvent system. Fractions containing earlier eluted product were combined and concentrated to obtain 1-[3-(6-chloro)-benzisoxazoyl]-5-trifluoromethoxylbenzimidazole-2-one as a white solid. ¹H NMR (DMSO, 500 MHz) δ 8.28 (d, J=9.0 Hz, 1H), 8.12 (s, 1H), 7.74 (d, J=9.0 Hz, 1H), 7.54 (d, J=8.5, 1H), 7.14 (d, J=8.5, 1H), 7.13 (s, 1H). Fractions containing later eluted product were combined and concentrated to obtain 1-[3-(6-chloro)-benzisoxazoyl]-6-trifluoromethoxylbenzimidazole-2-one as a white solid. ¹H NMR (DMSO, 500 MHz) δ 8.28 (d, J=9.0 Hz, 1H), 8.12 (s, 1H), 7.74 (d, J=9.0 Hz, 1H), 7.66 (brs, 1H), 7.54 (d, J=8.5, 1H), 7.13 (brs, 1H).

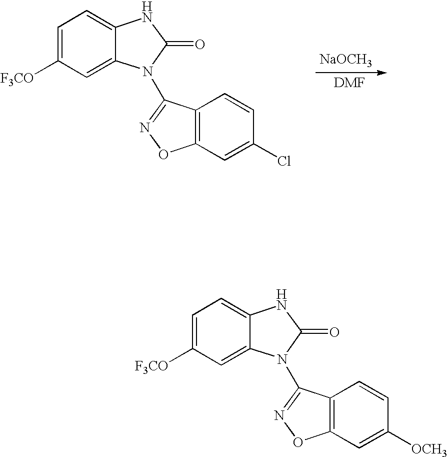

### 1-[(6-methoxy)-benzisoxazol-3-yl]-6-trifluoromethoxylbenzimidazole-2-one

To a solution of 1-[3-(6-chloro)-benzisoxazoyl]-6-trifluoromethoxylbenzimidazole-2-one (1.2 g, 3.25 mmol) in DMF (5 ml) was added NaOMe solution in methanol (30% wt/wt, 20 ml). The mixture was heated to 80° C. under vacuum to remove residual methanol and then stirred at 110° C. under nitrogen overnight. The mixture was then cooled to room temperature, diluted with water (50 ml), adjusted to pH 6 with 10% aqueous HCl. The resulting suspension was stirred in an ice bath for 2 hours and filtered. The solid was washed with water and dried in an oven (90° C.) for 2 hours and then under high vacuum at room temperature for 3 hours to obtain a slightly yellow solid. LC-MS m/e: (M+1)=366.

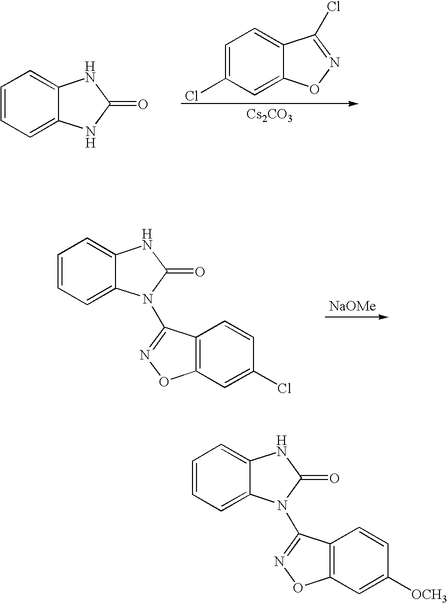

### 1-(6-chlorobenzisoxazol-3-yl)benzimidazole-2-one and 1-(6-methoxybenzisoxazol-3-yl)]benzimidazole-2-one

To a solution of benzimidazole-2-one (Aldrich, 1.1 g, 7.5 mmol) in DMF (20 ml) was added 3,6-dichlorobenzoisoxazole (1.5 g, 7.5 mmol) and Cs₂CO₃ (4.8 g, 15 mmol). The suspension was heated to 150° C. in an oil bath and stirred overnight. The mixture was then cooled to room temperature, diluted with water (30 ml) and stirred in a ice bath for 2 hours. The mixture was then filtered. The solid was washed with water and dried under high vacuum to obtain 2.0 g (93%) of a yellow solid as 1-(6-chlorobenzisoxazol-3-yl)benzimidazole-2-one. To a solution of the above product (1.0 g, 3.5 mmol) in DMF (15 ml) was added NaOMe solution in methanol (30% wt/wt, 10 ml). The mixture was heated to 80° C. under vacuum to remove residual methanol and then stirred at 110° C. under nitrogen overnight. The mixture was then cooled to room temperature, diluted with water (50 ml), adjusted to pH 6 with 10% aqueous HCl. The resulting suspension was stirred in an ice bath for 2 hours and filtered. The solid was washed with water and dried in an oven (90° C.) for 2 hours and then under high vacuum at room temperature for 3 hours to obtain a slightly yellow solid. LC-MS m/e: (M+1)=282

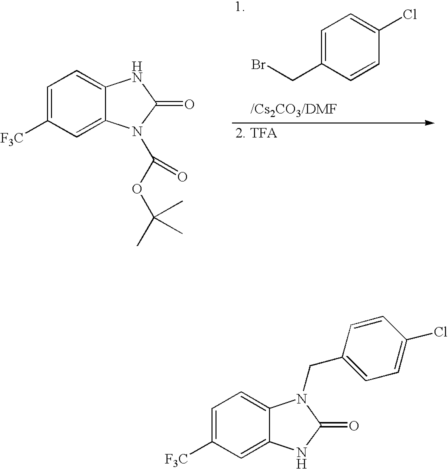

### 1-(4-chlorobenzyl)-5-trifluoromethylbenzimidazole-2-one

To the solution of 1-Boc-5-trifluoromethylbenzimidazole-2-one (100 mg, 0.33 mmol) in DMF (2 ml) was added 4-chlorobenzyl bromide (81 mg, 0.39 mmol) and Cs₂CO₃ (300 mg, 0.92 mmol). The mixture was stirred at room temperature for 2 hours, diluted with water (4 ml), and extracted with ethyl acetate (2×3 ml). The organic extracts were combined, and concentrated to obtain a solid. The solid was dissolved in TFA (1.5 ml), stirred at room temperature for 2 hours, and concentrated to dryness. The residue was purified by silica gel chromatography with hexane/ethyl acetate (4:1) as solvent system to obtain a white solid. ¹H NMR (DMSO, 500 MHz) δ 9.35 (brs, 1H), 7.38 (s, 1H), 7.34 (d, 2H), 7.28 (d, 2H), 7.27 (d, 1H), 6.93 (d, 1H), 5.10 (s. 2H).

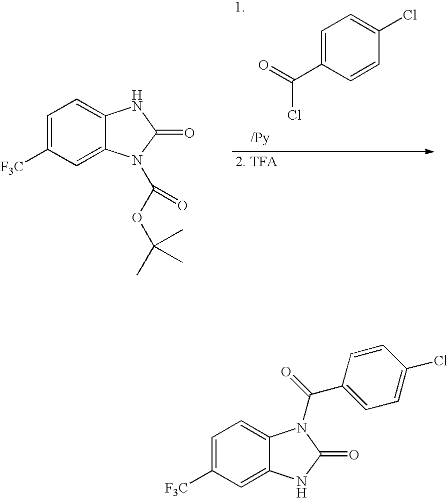

### 1-(4-chlorobenzoyl)-5-trifluoromethylbenzimidazole-2-one

To the solution of 1-Boc-5-trifluoromethylbenzimidazole-2-one (200 mg, 0.66 mmol) in anhydrous pyridine (4 ml) was added 4-chlorobenzoyl chloride (115 mg, 0.66 mmol). The mixture was stirred at room temperature overnight and concentrated to dryness. The residue was dissolved in TFA (2 ml), and stirred at room temperature for 2 hours. The mixture was concentrated to dryness and purified by silica gel column chromatography to obtain a solid. ¹H NMR (DMSO, 500 MHz) δ 7.92 (d, 1H), 7.81 (d, 2H), 7.56 (d, 2H), 7.66 (brs, 1H), 7.48 (d, 1H), 7.32 (brs, 1H).

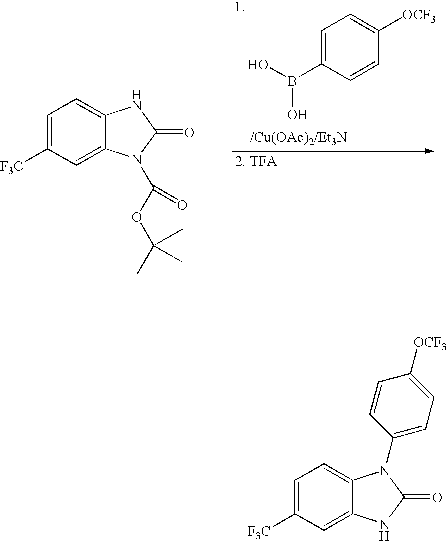

### 1-(4-trifluoromethoxyphenyl)-5-trifluoromethylbenzimidazole-2-one

To the solution of 1-Boc-5-trifluoromethylbenzimidazole-2-one (870 mg, 2.88 mmol) in methylene chloride (100 ml) was added Cu(OAc)₂ (521 mg, 2.88 mmol), triethylamine (0.94 ml, 14 mmol), and molecular sieves (200 mg). The mixture was stirred at room temperature under open air overnight, and filtered. The filtrate was concentrated to dryness. The residue was dissolved in TFA (5 ml), stirred at room temperature for 3 hours and concentrated to dryness. The residue was dissolved in ethyl acetate (5 ml) and passed through a silica gel pad and washed with hexane/ethyl acetate (3:1) (100 ml). The filtrate was concentrated to obtain a solid. LC-MS m/e (M+1)=363.

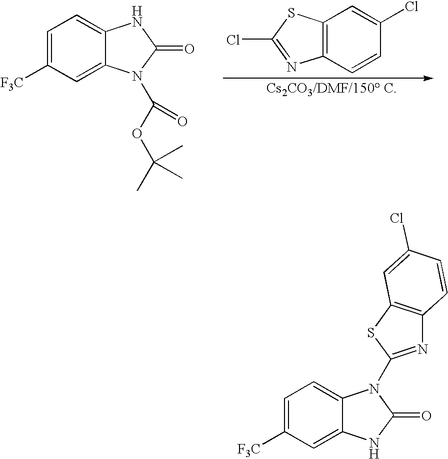

### 1-[(6-chloro)benzothiazol-2-yl]-5-trifluoromethylbenzimidazole-2-one

To the solution of 1-Boc-5-trifluoromethylbenzimidazole-2-one (500 mg, 1.65 mmol) in DMF (5 ml) was added 2,6-dichlorobenzothiazole (336 mg, 1.65 mmol) and Cs₂CO₃ (1.2 g, 3.68 mmol). The mixture was heated in an oil bath to 150° C., and stirred overnight. The reaction mixture was then cooled to room temperature, diluted with water (50 ml) and extracted with ethyl acetate (2×20 ml). The organic extracts were combined, concentrated to dryness, and purified by silica gel column chromatography with hexane/ethyl acetate (3:1) as solvent system to obtain a white powder. LC-MS m/e (M+1)=372.

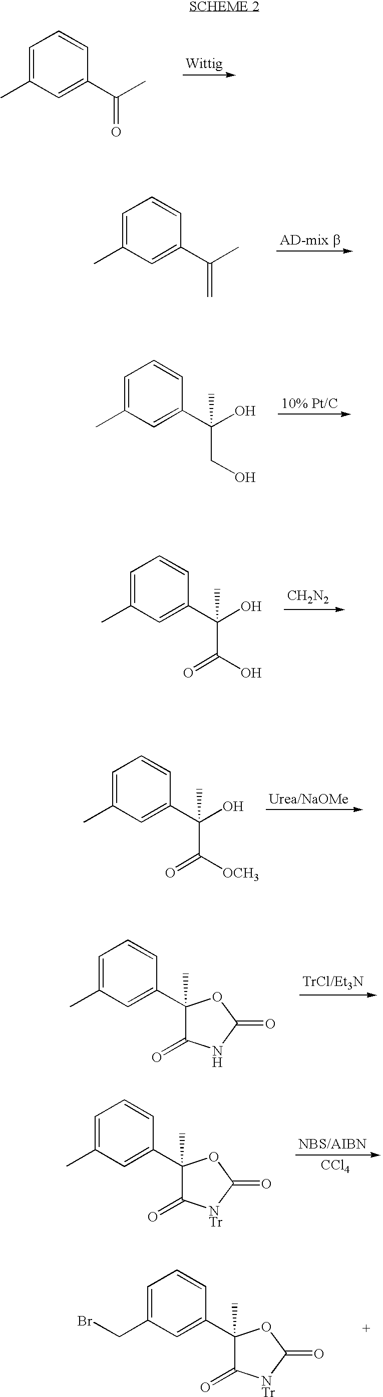

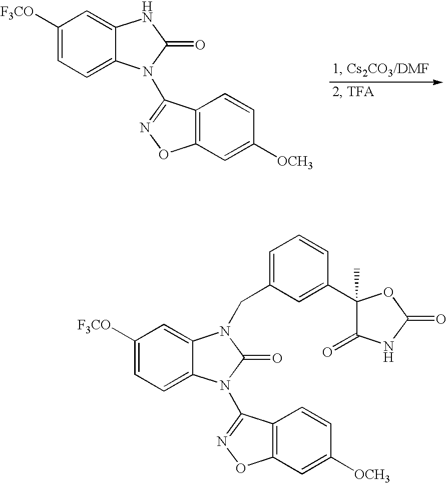

### Experimental Procedures:

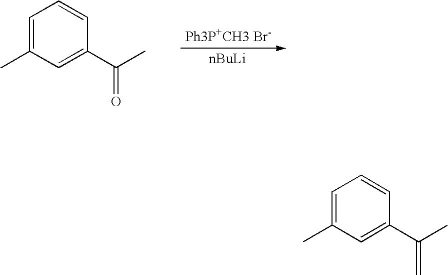

### 2-(3-Methylphenyl)propene

To a suspension of methyltriphenylphosphonium bromide (33.2 g, 92.9 mmol) in diethyl ether (200 mL) at room temperature was added slowly nBuLi (2.5 M in hexanes, 37 mL). The resulting yellow solution was stirred for 30 min before 3-methylacetophenone (12 mL, 90 mmol) was introduced. The reaction was stirred at room temperature overnight. The precipitate was removed by filtration. The solvent was removed in vacuum. Purification by flash chromatography gave the 2-(3-methylphenyl)propene product.

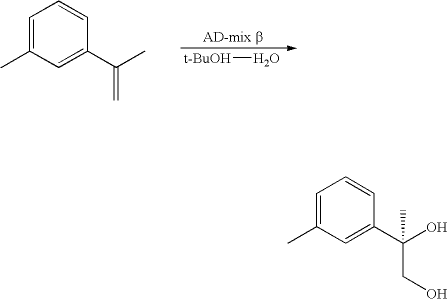

### 2(R)-(3-Methylphenyl)-1,2-propane-diol

To a suspension of AD-mix-β (7 g) in H₂O/tBuOH (25 mL/25 mL) at 0° C. was added 2-(3-Methylphenyl)propene (0.66 g, 5 mmol). The reaction mixture was stirred at 0° C. overnight. Na₂SO₃ (7.5 g) was added. After 1 h at room temperature, the mixture was extracted with EtOAc (3×). The combined organic layers were washed with brine, dried over Na₂SO₄, filtered, and concentrated in vacuum. Purification by flash chromatography gave 2(R)-(3-methylphenyl)-1,2-propane-diol ¹H NMR (500 MHz, CDCl₃) δ 7.30 (overlapping signals, 2H), 7.12 (d, J=7.1 Hz, 1H), 3.82 (d, J=11.2 Hz), 3.66 (d, J=11.2 Hz, 1H), 2.59 (s, 1H), 2.40 (s, 3H), 1.55 (s, 3H).

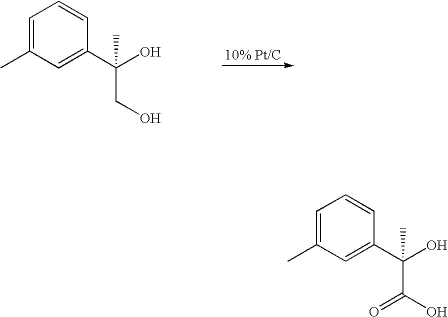

### 2(R)-(3-Methylphenyl)lactic acid

To a solution of 2(R)-(3-Methylphenyl)-1,2-propane-diol (0.7 g) in water (50 mL) were added NaHCO₃ (0.4 g) and 10% Pt/C (0.7 g). Air was bubbled through the reaction mixture via a gas dispenser at 70° C. overnight. The reaction was cooled to room temperature, then filtered through Celite. The filtrate was acidified with aqueous H₂SO₄ (1.0 N) to pH 2, then extracted with EtOAc (3×). The combined organic layers were washed with brine, dried over Na₂SO₄, filtered, and concentrated in vacuum to give the acid. ¹HNMR (600 MHz, DMSO) δ(ppm): 7.32 (s, 1H), 7.28 (d, 1H), 7.19 (t, 1H), 7.02 (d, 1H), 2.25 (s, 3H), 1.59 (s, 3H).

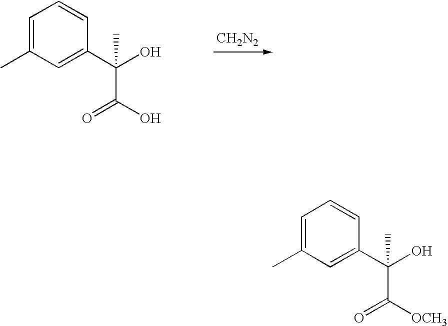

### Methyl 2(R)-(3-methylphenyl)lactate

To a solution of 2(R)-(3-methylphenyl)lactic acid (3.64 g, 20.2 mmol) in anhydrous diethyl ether (100 ml) was added diazomethane ether solution (freshly produced following procedures in Aldrich Technical Bulletin AL-180) until a bright yellow color is produced or no more bubbles were evolved. The solution was then concentrated to dryness to a white solid. ¹HNMR (600 MHz, CDCl3) δ(ppm): 7.39 (s, 1H), 7.35 (d, 1H), 7.25 (t, 1H), 7.13 (d, 1H), 3.79 (s, 3H), 2.40 (s, 3H), 1.80 (s, 3H).

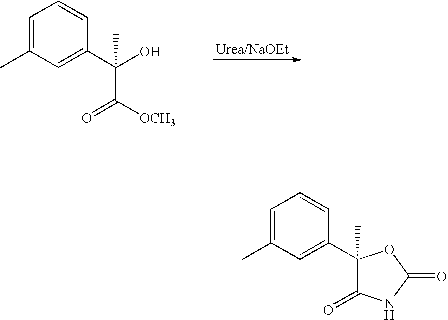

### (R)-5-Methyl-5-(3-methylphenyl)oxazolidinedione

To a solution of methyl 2(R)-(3-methylphenyl)lactate (3.9 g, 20.1 mmol) in anhydrous ethanol (50 ml), was added NaOEt in ethanol (21% wt/wt, 9.8 ml, 30 mmol), and urea (1.5 g, 24.2 mmol). The mixture was heated to 95° C. and refluxed overnight. The solution was then cooled to room temperature, acidified with 1N HCl, concentrated to small volume, and diluted with water (100 ml). The aqueous mixture was extracted with ethyl acetate (3×50 ml). The organic extracts were combined, washed with brine, dried over anhydrous Mg₂SO₄, and concentrated to dryness to obtain an oil which was used in the next step without further purification. ¹H NMR (500 MHz, CDCl₃) δ 8.70 (s, broad, 1H), 7.4-7.2 (overlapping signals, 4H), 2.40 (s, 3H), 1.96 (s, 3H).

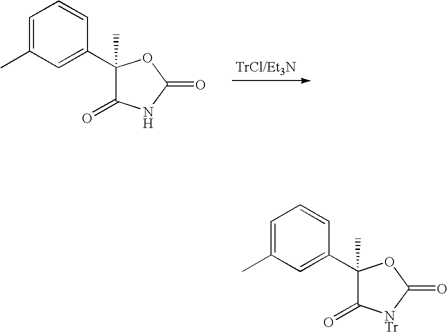

### (R)-5-Methyl-5-(3-methylphenyl)-N-trityloxazolidinedione

To a solution of (R)-5-methyl-5-(3-methylphenyl)oxazolindione (4.3 g, 20.9 mmol) in anhydrous methylene chloride (50 ml) was added triethyl amine (2.1 g, 23 mmol) and trityl chloride (6.4 g, 23 mmol). The mixture was stirred under nitrogen at room temperature for 1 hour, washed with water (20 ml), brine (20 ml), dried over anhydrous Mg₂SO₄, and concentrated to dryness to obtain a solid. ¹H NMR (500 MHz, CDCl₃) δ 7.40-7.20 (multiple overlapping peaks, 19H), 2.40 (s, 3H), 1.76 (s, 3H).

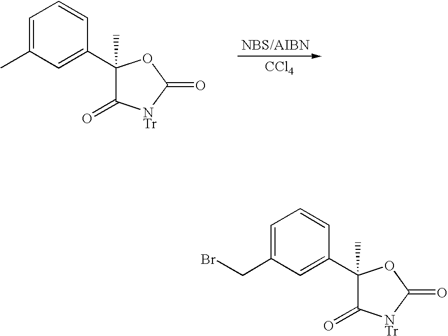

### (R)-5-Bromomethyl-5-(3-methylphenyl)-N-trityloxazolidinedione

To a solution of (R)-5-methyl-5-(3-methylphenyl)-N-trityloxazolidinedione (3.0 g, 6.7 mmol) in carbon tetrachloride (100 ml) was added N-bromosuccinamide (1.1 g, 6.7 mmol), and AIBN (catalytic). The mixture was heated to 80° C. and refluxed overnight. The solution was then cooled to room temperature, washed with saturated NaHCO₃ solution (20 ml), water (20 ml), and brine (20 ml), and concentrated to dryness. The residue was purified by silica gel column chromatography with hexane/ethyl acetate (9:10) as a solvent to obtain a white solid. ¹H NMR (500 MHz, CDCl₃) δ 7.5-7.2 (multiple overlapping peaks, 19H), 4.56 (s, 2HH), 1.76 (s, 3H).

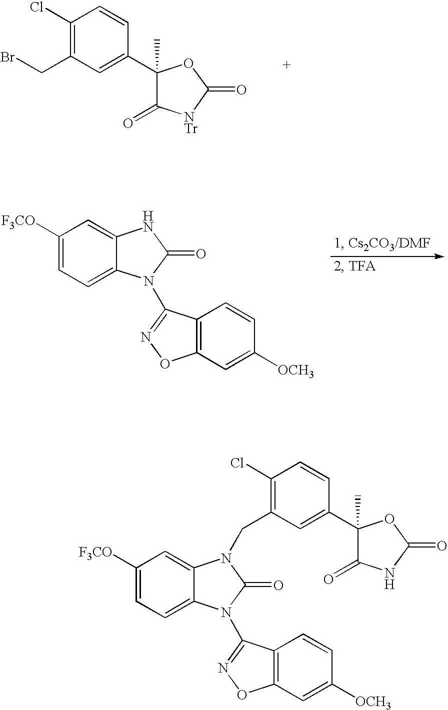

### (5R)-5-(4-chloro-3-{[3-(6-methoxy-1,2-benzisoxazol-3-yl)-2-oxo-6-(trifluoromethoxy)-2,3-dihydro-1H-benzimidazol-1-yl]methyl}phenyl)-5-methyl-1,3-oxazolidine-2,4-dione

To a solution of 1-[3-(6-methoxy)-benzisoxazoyl]-5-trifluoromethoxylbenzimidazole-2-one (700 mg, 1.92 mmol) in DMF (20 ml) were added (R)-5-methyl-5-(3-bromomethyl-4-chlorophenyl)-N-trityloxazolidinedione (1.1 g, 1.93 mmol), and Cs₂CO₃ (1.25 g, 3.8 mmol). The mixture was stirred at room temperature overnight, diluted with water (30 ml) and extracted with ethyl acetate (2×30 ml). The organic extracts were combined, dried over anhydrous Mg₂SO₄, and concentrated to obtain 1.5 g (92%) of a solid. The solid was dissolved in neat trifluoromethane sulfonic acid (5 ml) and stirred at room temperature for 6 hours. The mixture was then concentrated to dryness under vacuum, and purified by silica gel column chromatography with hexane/ethyl acetate/TFA (3/1/0.01) as solvent system to obtain a white solid. ¹H NMR (500 MHz, CDCl₃) δ 8.59 (brs, 1H), 8.14 (d, 1H), 7.95 (d, 1H), 7.62 (s, 1H), 7.56 (s, 2H), 7.03 (m, 3H), 6.91(s, 1H), 5.34 (s, 2H), 3.98 (s, 3H), 1.84 (s, 3H).

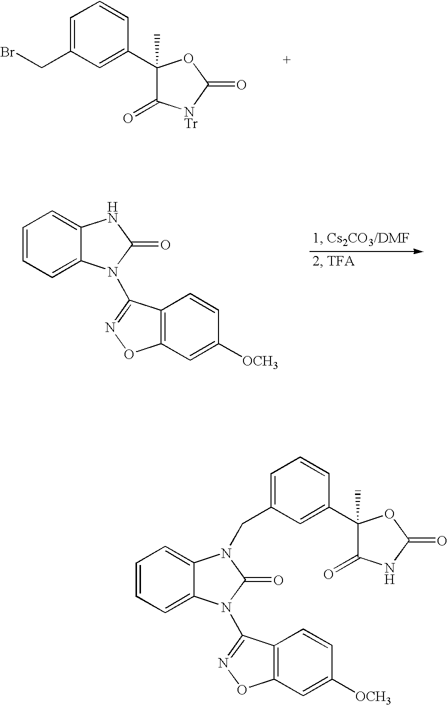

### (5R)-5-(3-{[3-(6-methoxy-1,2-benzisoxazol-3-yl)-2-oxo-2,3-dihydro-1H-benzimidazol-1-yl]methyl}phenyl)-5-methyl-1,3-oxazolidine-2,4-dione

To a solution of 1-[3-(6-methoxy)-benzisoxazoyl]benzimidazole-2-one (266 mg, 0.95 mmol) in DMF (3 ml) were added (R)-5-methyl-5-(3-bromomethylphenyl)-N-trityloxazolidinedione (500 mg, 0.95 mmol), and Cs₂CO₃ (620 mg, 1.9 mmol). The mixture was stirred at room temperature overnight, diluted with water (10 ml) and extracted with ethyl acetate (2×10 ml). The organic extracts were combined, dried over anhydrous Mg₂SO₄, and concentrated to obtain a solid. The solid was dissolved in neat trifluoromethane sulfonic acid (5 ml) and stirred at room temperature for 6 hours. The mixture was then concentrated to dryness under vacuum, and purified by silica gel column chromatography with hexane/ethyl acetate/TFA (3/1/0.01) as solvent system to obtain a white solid. ¹H NMR (500 MHz, CDCl₃) δ 8.18 (d, 1H), 7.92 (d, 1H), 7.71 (s, 1H), 7.56 (brs, 1H), 7.43 (brs, 2H), 7.23 (m, 2H), 7.08 (s, 1H), 7.02 (m, 2H), 5.28 (dd, 2H), 3.98 (s, 3H), 1.99 (s, 3H).

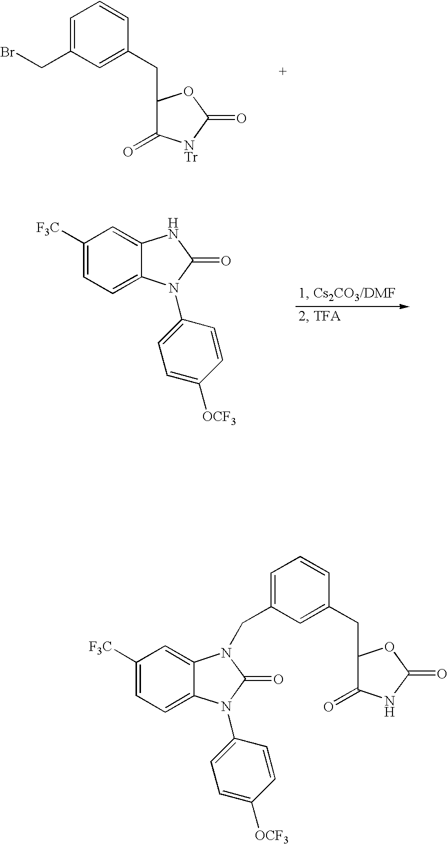

### 5-(3-{[2-oxo-3-[4-(trifluoromethoxy)phenyl]-6-(trifluoromethyl)-2,3-dihydro-1H-benzimidazol-1-yl]methyl}benzyl)-1,3-oxazolidine-2,4-dione

To the solution of 1-(4-trifluoromethoxyphenyl)-5-trifluoromethylbenzimidazole-2-one (362 mg, 1 mmol) in DMF (4 ml) was added 5-(3-bromomethylbenzyl)-N-trityloxazolidinedione (525 mg, 1.0 mmol), and Cs₂CO₃ (620 mg, 1.9 mmol). The mixture was stirred at room temperature overnight, diluted with water (10 ml) and extracted with ethyl acetate (2×10 ml). The organic extracts were combined, dried over anhydrous Mg₂SO₄, and concentrated to obtain a solid. The solid was dissolved in neat trifluoromethane sulfonic acid (2 ml) and stirred at room temperature for 4 hours. The mixture was then concentrated to dryness under vacuum, and purified by silica gel column chromatography with hexane/ethyl acetate/TFA (3/1/0.01) as solvent system to obtain a white solid. LC-MS m/e: (M+1)=565

### EXAMPLES

### Example 1

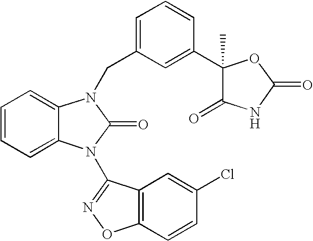

### (5R)-5-(3-{[3-(5-chloro-1,2-benzisoxazol-3-yl)-2-oxo-2,3-dihydro-1H-benzimidazol-1-yl]methyl}phenyl)-5-methyl-1,3-oxazolidine-2,4-dione

¹H NMR (500 MHz, CDCl₃) δ 8.39 (s, 1H), 8.33 (brs, 1H), 7.92 (d, 1H), 7.71 (s, 1H), 7.61 (s, 2H), 7.58 (d, 1H), 7.42 (m, 2H), 7.21 (d, 2H), 7.02(d, 1H), 5.23 (dd, 2H), 1.99 (s, 3H).

### Example 2

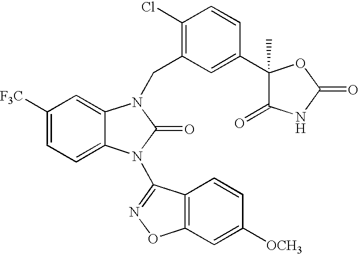

### (5R)-5-(4-chloro-3-{[3-(6-methoxy-1,2-benzisoxazol-3-yl)-2-oxo-6-(trifluoromethyl)-2,3-dihydro-1H-benzimidazol-1-yl]methyl}phenyl)-5-methyl-1,3-oxazolidine-2,4-dione

¹H NMR (500 MHz, CDCl₃) δ 8.14 (d, 1H), 8.02 (d, 1H), 7.67 (s, 1H), 7.57 (s, 1H), 7.52 (d, 1H), 7.26 (m, 2H), 7.09 (s, 1H), 7.06 (d, 1H), 5.38 (s, 2H), 3.98 (s, 3H), 1.83 (s, 3H).

### Example 3

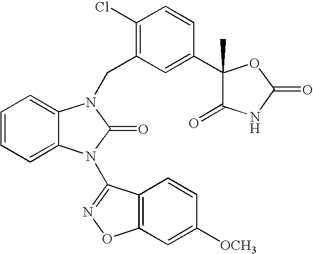

### (5S)-5-(4-chloro-3-{[3-(6-methoxy-1,2-benzisoxazol-3-yl)-2-oxo-2,3-dihydro-1H-benzimidazol-1-yl]methyl}phenyl)-5-methyl-1,3-oxazolidine-2,4-dione

¹H NMR (500 MHz, CDCl₃) δ 9.30 (brs, 1H), 8.09 (d, 1H), 7.88 (d, 1H), 7.61 (s, 1H), 7.42 (s, 2H), 7.21 (m, 2H), 7.01 (m, 3H), 5.32 (s, 2H), 3.98 (s, 3H), 1.76 (s, 3H).

### Example 4

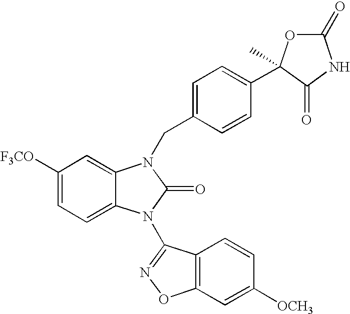

### (5R)-5-(4-{[3-(6-methoxy-1,2-benzisoxazol-3-yl)-2-oxo-6-(trifluoromethoxy)-2,3-dihydro-1H-benzimidazol-1-yl]methyl}phenyl)-5-methyl-1,3-oxazolidine-2,4-dione

¹H NMR (500 MHz, CDCl₃) δ 8.19 (d, 1H), 7.98 (d, 1H), 7.65 (d, 2H), 7.52 (d, 2H), 7.13 (d, 1H), 7.11 (s, 1H), 7.02 (d, 1H), 6.83 (s, 1H), 5.20 (s, 2H), 3.98 (s, 3H), 1.99 (s, 3H).

### Example 5

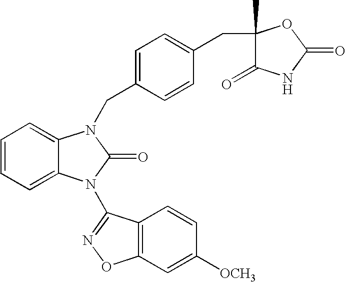

### (5S)-5-(4-{[3-(6-methoxy-1,2-benzisoxazol-3-yl)-2-oxo-2,3-dihydro-1H-benzimidazol-1-yl]methyl}benzyl)-5-methyl-1,3-oxazolidine-2,4-dione

¹H NMR (500 MHz, CDCl₃) δ 8.19 (d, 1H), 7.90 (d, 1H), 7.40 (d, 2H), 7.23 (d, 2H), 7.21 (m, 2H), 7.05 (s, 1H), 7.00 (d, 1H), 6.98 (d, 1H), 5.19 (dd, 2H), 3.98 (s, 3H), 3.18 (dd, 2H), 1.64 (s, 3H).

### Example 6

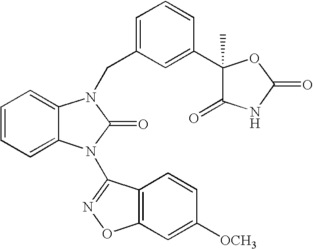

### (5R)-5-(3-{[3-(6-methoxy-1,2-benzisoxazol-3-yl)-2-oxo-2,3-dihydro-1H-benzimidazol-1-yl]methyl}phenyl)-5-methyl-1,3-oxazolidine-2,4-dione

1H NMR (500 MHz, CDCl3) δ 8.18 (d, 1H), 7.92 (d, 1H), 7.71 (s, 1H), 7.56 (brs, 1H), 7.43 (brs, 2H), 7.23 (m, 2H), 7.08 (s, 1H), 7.02 (m, 2H), 5.28 (dd, 2H), 3.98 (s, 3H), 1.99 (s, 3H).

Examples of other compounds of this invention are shown in Table 1. These compounds were made or can be made using the procedures disclosed herein.

TABLE 1

| | LC-MS | | LC-MS |
|---|---|---|---|
| Structure | (M + 1) | | (M + 1) |
| 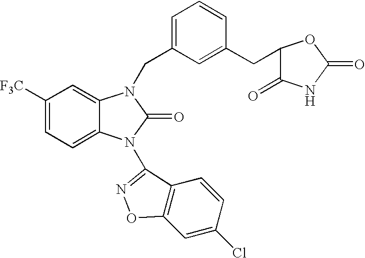 | 557 | 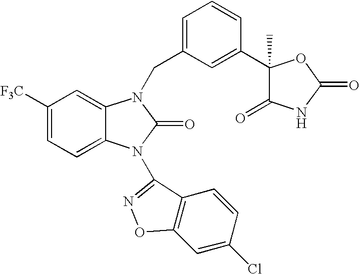 | 557 |
| 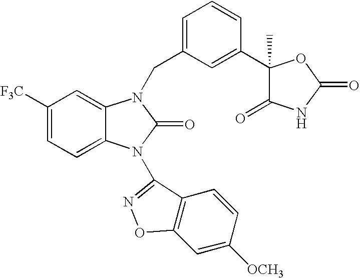 | 553 | 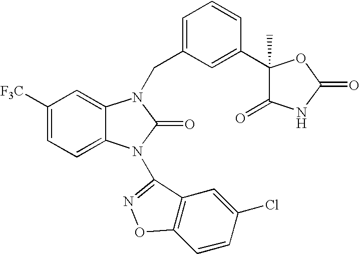 | 557 |
| 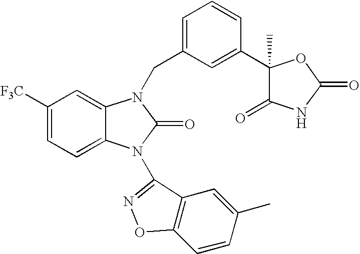 | 537 | 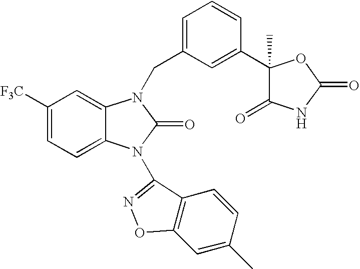 | 537 |
| 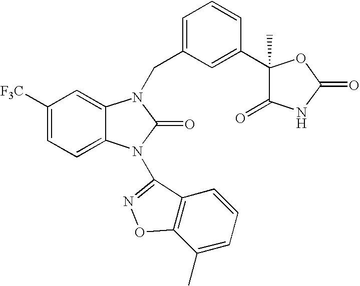 | 537 | 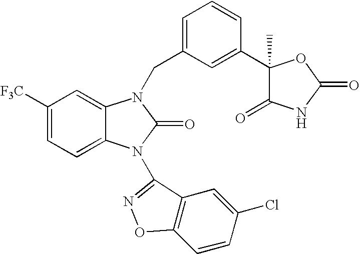 | 557 |
| 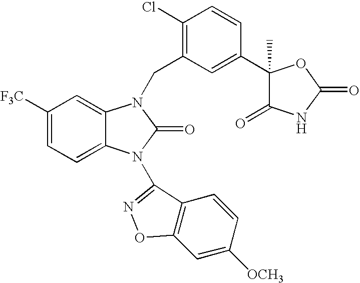 | 587 | 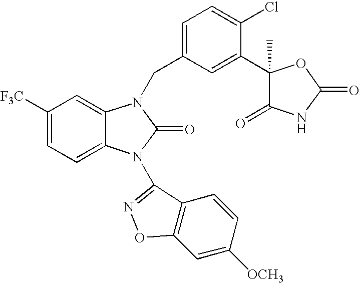 | 587 |
| 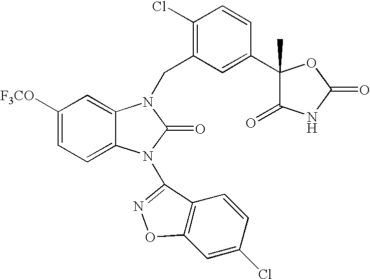 | 607 | 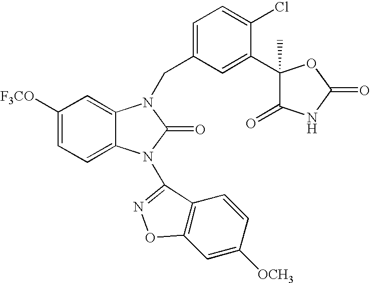 | 603 |
| 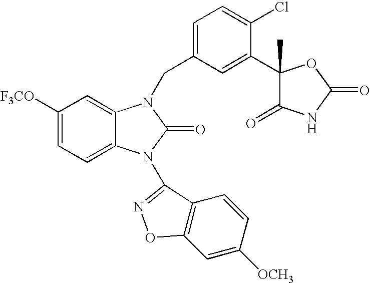 | 603 | 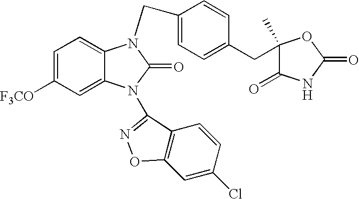 | 587 |
| 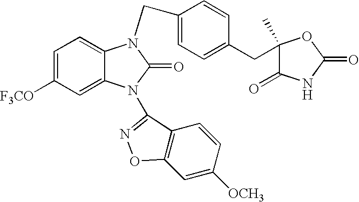 | 583 | 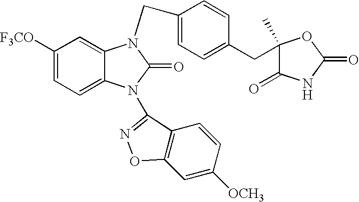 | 583 |
| 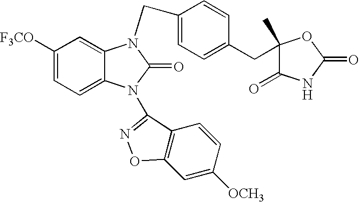 | 583 | 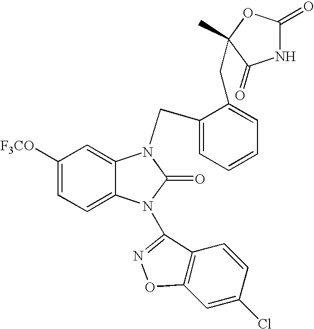 | 587 |
| 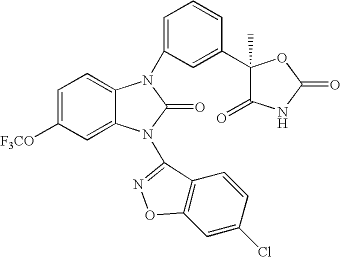 | 559 | 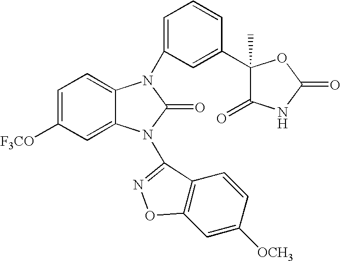 | 555 |
| 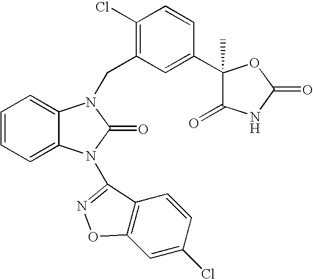 | 523 | 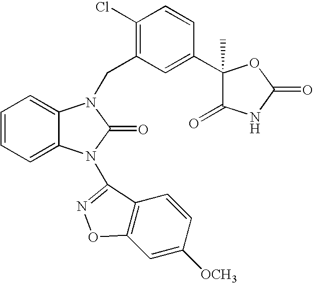 | 519 |
| 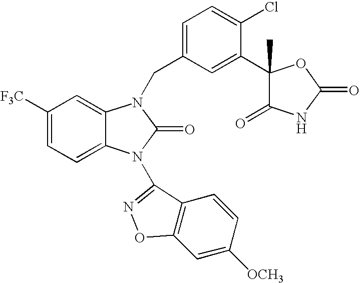 | 587 | 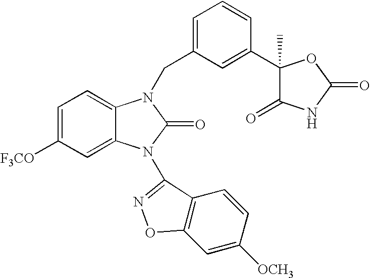 | 569 |
| 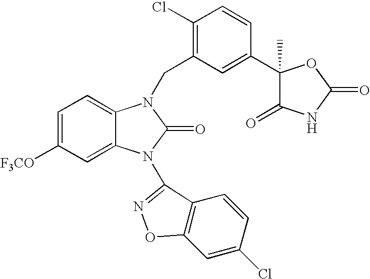 | 607 | 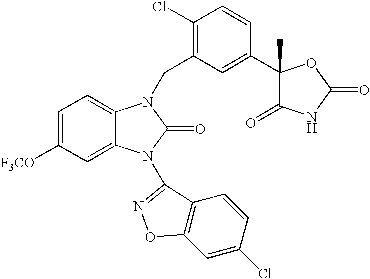 | 607 |
| 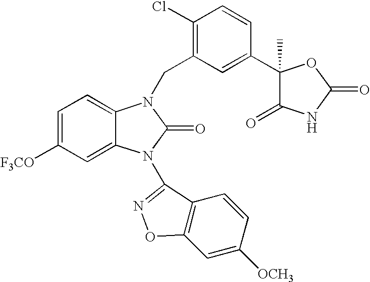 | 603 | 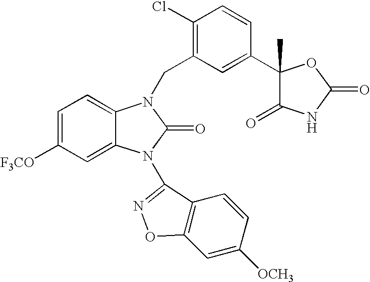 | 603 |
| 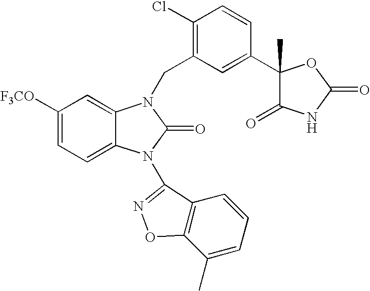 | 587 | 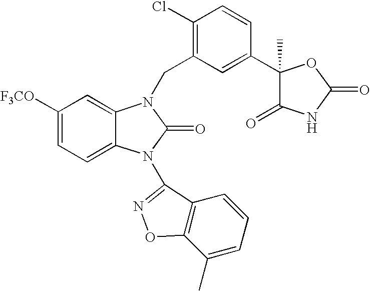 | 587 |
| 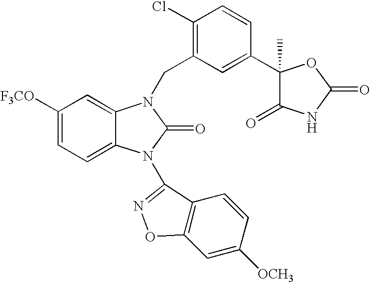 | 603 | 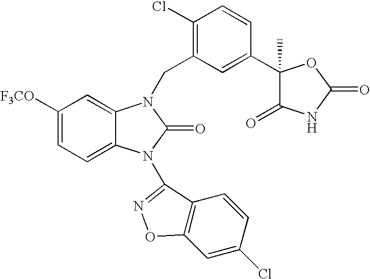 | 607 |
| 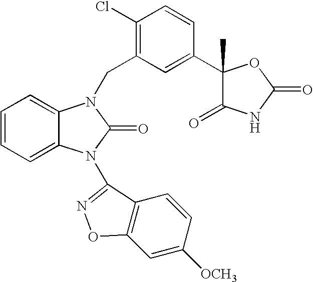 | 519 | 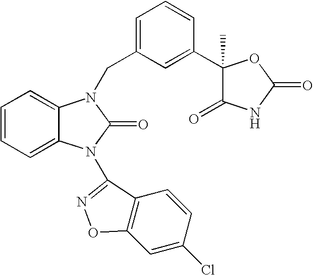 | 489 |
| 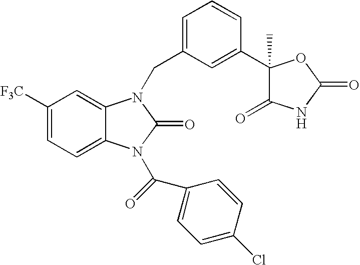 | | 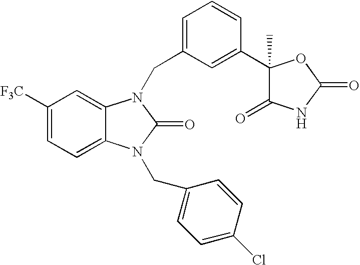 | |
| 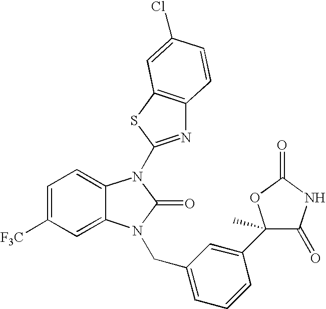 | | 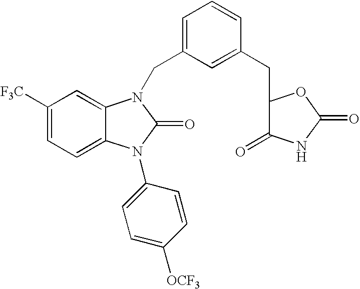 | 566 |

1. A compound of formula I: 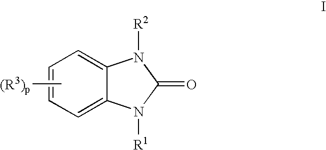
or a pharmaceutically acceptable salt thereof, wherein:
R¹ is —X-Aryl-Y-Z, wherein Aryl is optionally substituted with 1-3 groups independently selected from A;
Aryl is phenyl or naphthyl;
X and Y are each independently selected from the group consisting of a bond and —CR⁴R⁵—;
Z is 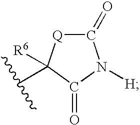
Q is selected from the group consisting of S and O;
A is selected from the group consisting of C₁-C₄ alkyl, C₂-C₄ alkenyl, —OC₁-C₄ alkyl, and halogen, wherein alkyl, alkenyl, and —Oalkyl are each optionally substituted with 1-5 halogens;
R² is selected from the group consisting of
(a) Benzisoxazolyl,
(b) Aryl,
(c) —(CH₂)Aryl,
(d) —(C═O)Aryl, and
(e) benzothiazolyl, wherein
R² is optionally substituted with 1-3 substituent groups independently selected from halogen, C₁-C₃ alkyl, and —OC₁-C₃ alkyl, wherein C₁-C₃ alkyl and —OC₁-C₃ alkyl are optionally substituted with 1-5 halogens;
R³ is selected from the group consisting of halogen, C₁-C₃ alkyl, and —OC₁-C₃ alkyl, wherein C₁-C₃ alkyl and —OC₁-C₃ alkyl are optionally substituted with 1-5 halogens;
R⁴ and R⁵ are each independently selected from the group consisting of hydrogen, halogen, C₁-C₃ alkyl, and —OC₁-C₃ alkyl, wherein C₁-C₃ alkyl and —OC₁-C₃ alkyl are optionally substituted with 1-5 halogens;
R⁶ is selected from the group consisting of H, C₁-C₃ alkyl, and halogen, wherein C₁-C₃ alkyl is optionally substituted with 1-3F; and
p is an integer from 0 to 4.

2. The compound of claim 1, or a pharmaceutically acceptable salt thereof, wherein R¹ is —X-phenyl-YZ.

3. The compound of claim 1, or a pharmaceutically acceptable salt thereof, wherein X and Y are each independently selected from a bond and —CH₂—.

4. The compound of claim 1, or a pharmaceutically acceptable salt thereof, wherein Aryl is optionally substituted with 1-2 groups independently selected from the group consisting of halogen, —CF₃, —OCF₃, —CH₃, and —OCH₃.

5. The compound of claim 1, or a pharmaceutically acceptable salt thereof, wherein
R³ is selected from the group consisting of —CH₃, —OCH₃, —OCF₃, and —CF₃;
R⁶ is selected from H, CH₃, and CF₃; and
p is 0 or 1.

6. The compound of claim 1, or a pharmaceutically acceptable salt thereof, wherein
R¹ is —X-phenyl-YZ, wherein phenyl is optionally substituted with 1-2 groups independently selected from A;
X and Y are each independently selected from a bond and —CH₂—;
A is selected from the group consisting of halogen, —CF₃, —OCF₃, —CH₃, and —OCH₃;
R³ is selected from the group consisting of —CF₃, —OCF₃, —CH₃, and —OCH₃;
R⁶ is selected from the group consisting of H, —CH₃, and —CF₃; and
p is 0 or 1.

7. The compound of claim 6, or a pharmaceutically acceptable salt thereof, wherein R² is 3-Benzisoxazolyl, which is optionally substituted with 1-2 groups independently selected from halogen, —OCH₃, —OCF₃, CH₃, and CF₃.

8. The compound of claim 1, or a pharmaceutically acceptable salt thereof, wherein Q is O, X is a bond, and Y is —CH₂—.

9. The compound of claim 1, or a pharmaceutically acceptable salt thereof, wherein Q is O, X is —CH₂—, and Y is a bond.

10. The compound of claim 1, or a pharmaceutically acceptable salt thereof, wherein Q is O and X and Y are each —CH₂—.

11. The compound of claim 1, or a pharmaceutically acceptable salt thereof, wherein Q is O, and X and Y are each a bond.

12. The compound of claim 1, or a pharmaceutically acceptable salt thereof, wherein Q is S.

13. The compound of claim 1 having a structure selected from the group consisting of the structures below, or a pharmaceutically acceptable salt thereof:

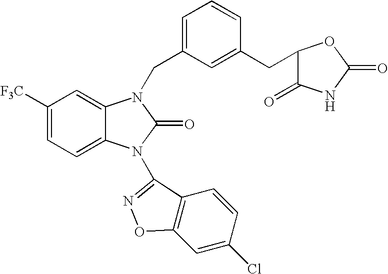

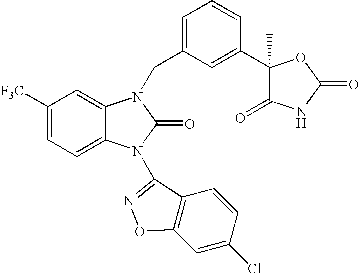

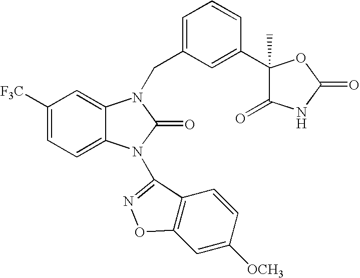

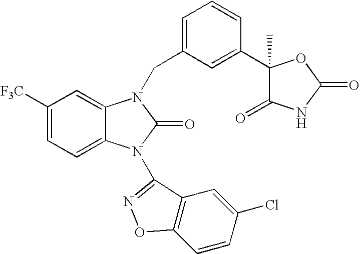

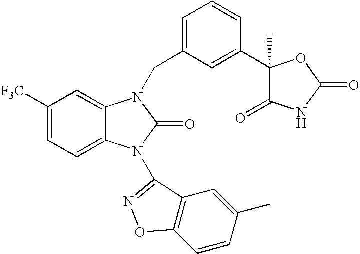

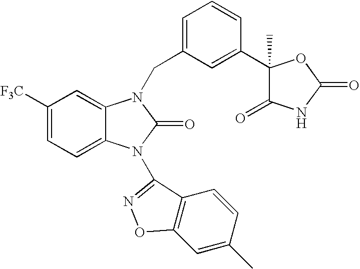

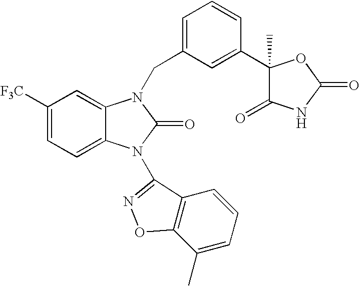

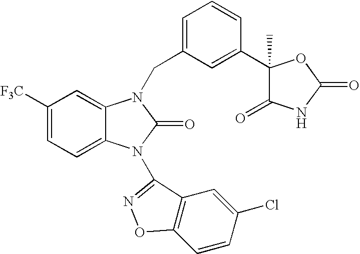

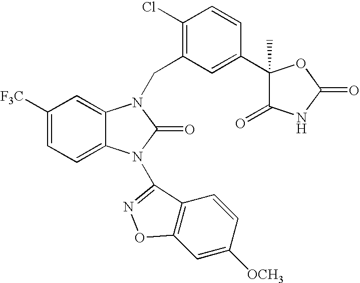

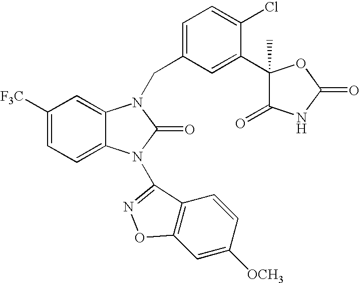

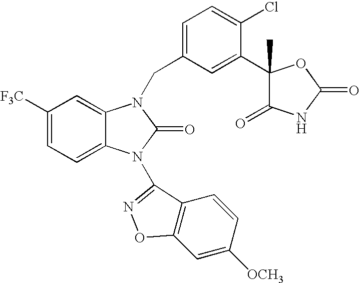

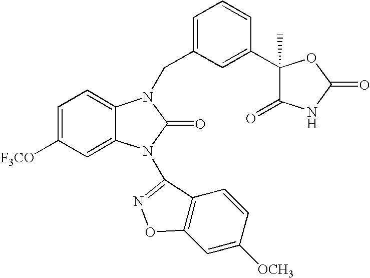

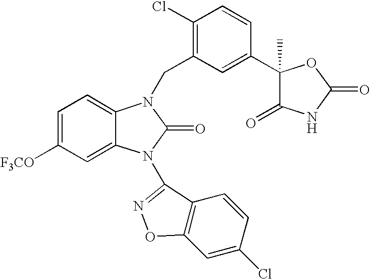

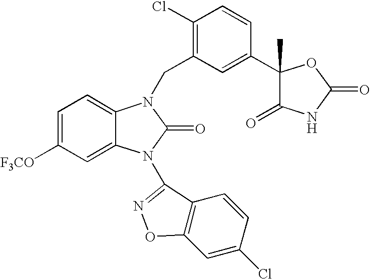

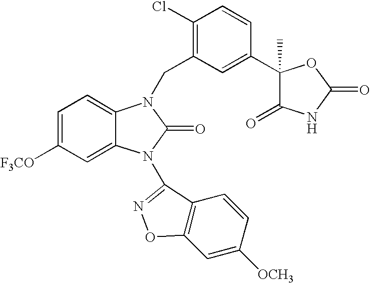

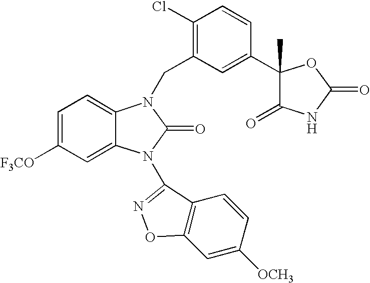

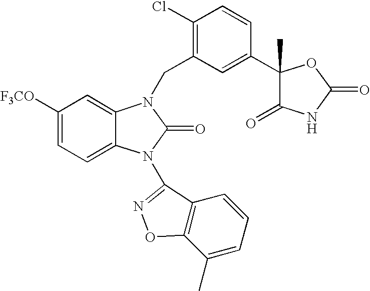

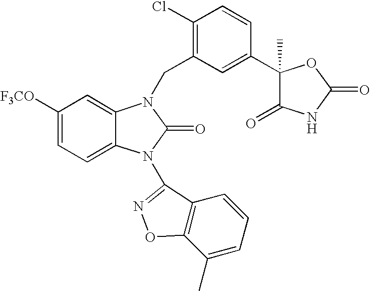

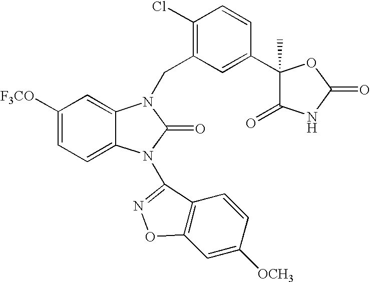

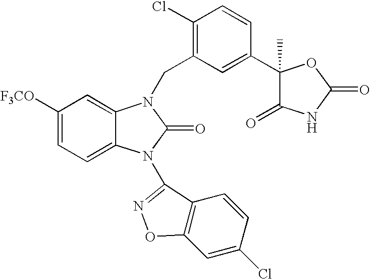

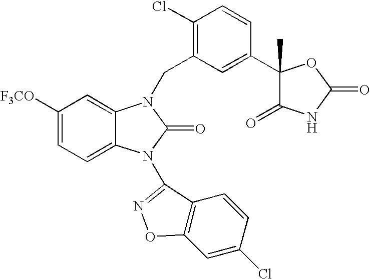

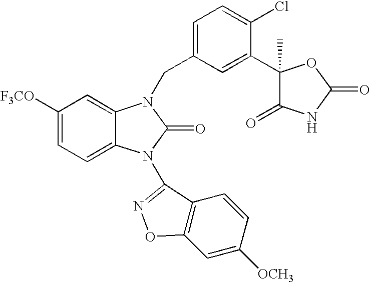

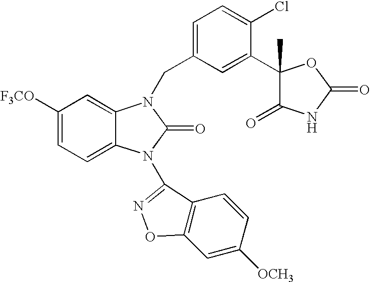

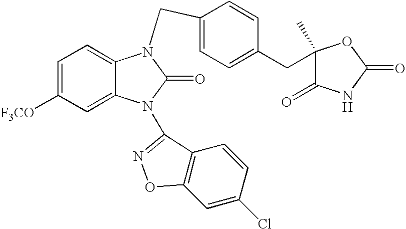

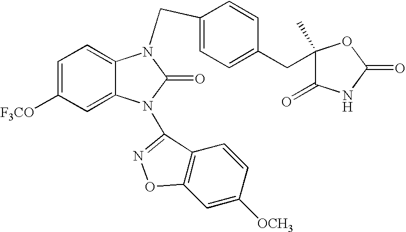

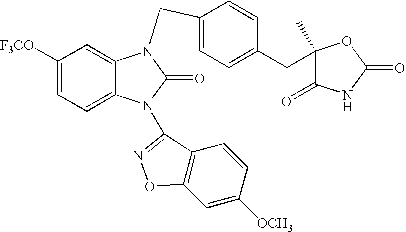

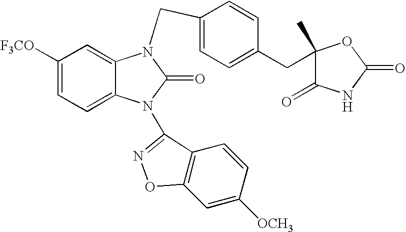

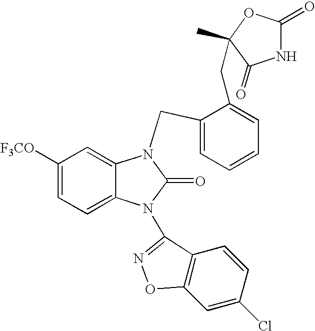

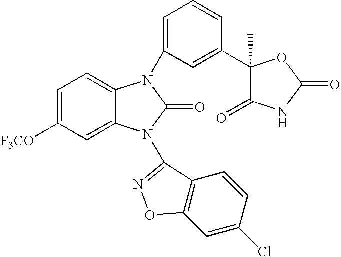

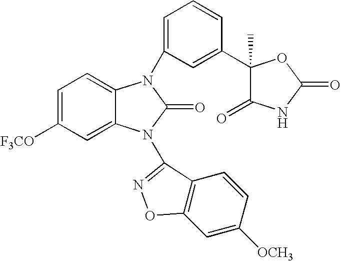

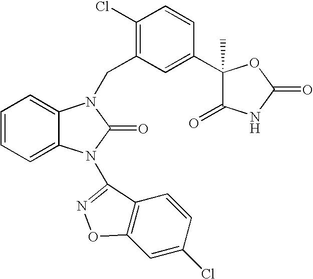

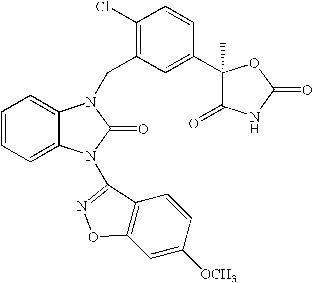

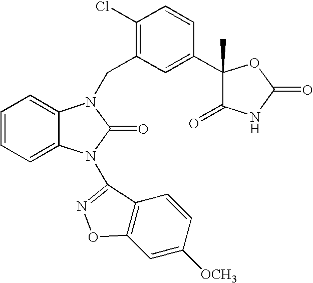

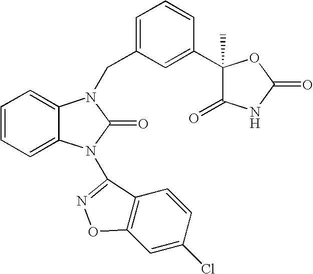

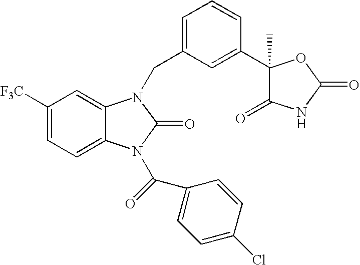

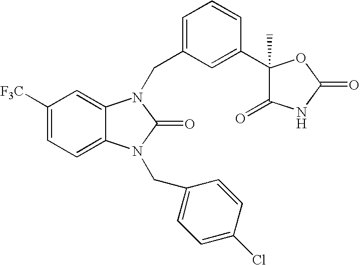

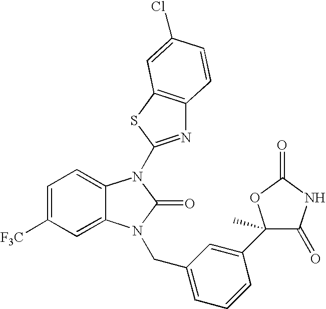

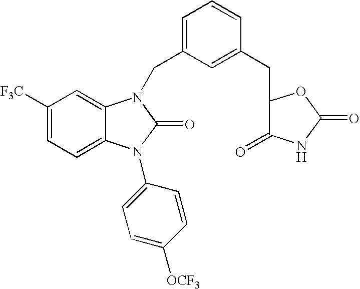

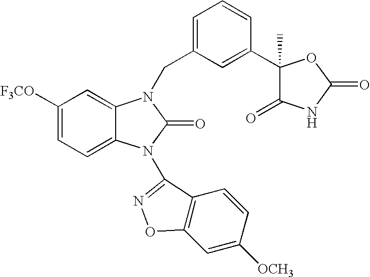

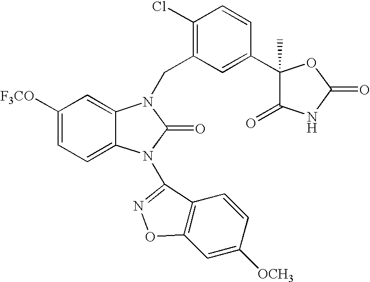

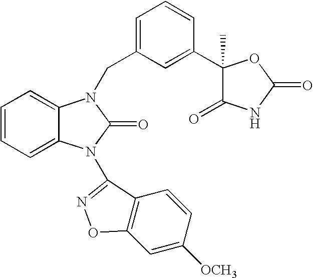

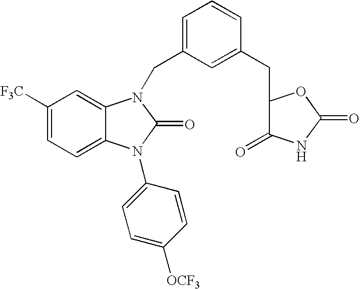

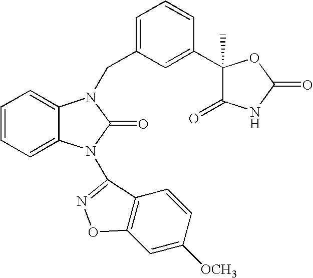

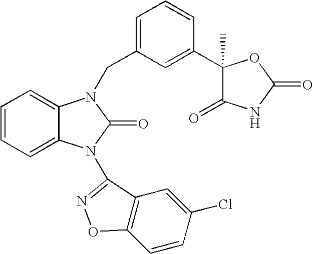

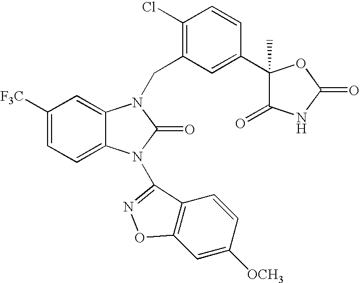

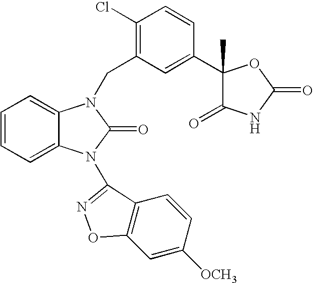

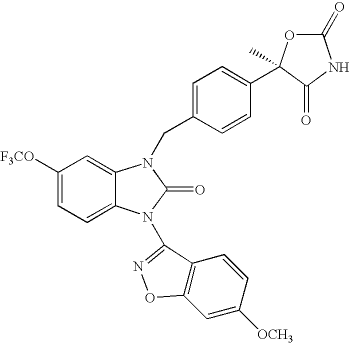

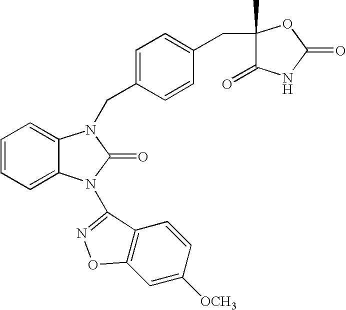

14. A pharmaceutical composition comprising a compound of claim 1, or a pharmaceutically acceptable salt thereof, and a pharmaceutically acceptable carrier.

15. (canceled)

16. A method of treating one or more diseases, disorders, or conditions selected from the group consisting of (1) non-insulin dependent diabetes mellitus (NIDDM), (2) hyperglycemia, (3) low glucose tolerance, (4) insulin resistance, (5) obesity, (6) lipid disorders, (7) dyslipidemia, (8) hyperlipidemia, (9) hypertriglyceridemia, (10) hypercholesterolemia, (11) low HDL levels, (12) high LDL levels, (13) atherosclerosis and its sequelae, (14) vascular restenosis, (15) irritable bowel syndrome, (16) inflammatory bowel disease, (17) Crohn's disease, (18) ulcerative colitis, (19) abdominal obesity, (20) retinopathy, (21) psoriasis, (22) high blood pressure, (23) metabolic syndrome, (24) ovarian hyperandrogenism (polycystic ovarian syndrome), and other diseases, disorders or conditions where insulin resistance is a component, said method comprising the administration of an effective amount of a compound of claim 1, or a pharmaceutically acceptable salt thereof.

17. A method for treating non-insulin dependent (Type 2) diabetes mellitus in a patient in need of such treatment which comprises administering to said patient a therapeutically effective amount of a compound of claim 1, or a pharmaceutically acceptable salt thereof.

18. A method for treating diabetic dyslipidemia in a patient in need of such treatment which comprises administering to said patient a therapeutically effective amount of a compound of claim 1, or a pharmaceutically acceptable salt thereof.

19. A pharmaceutical composition comprising
(1) a compound of claim 1, or a pharmaceutically acceptable salt thereof;
(2) one or more compounds selected from the group consisting of:
(a) PPAR gamma agonists and partial agonists;
(b) biguanides;
(c) protein tyrosine phosphatase-1B (PTP-1B) inhibitors;
(d) dipeptidyl peptidase IV (DP-IV) inhibitors;
(e) insulin or an insulin mimetic;
(f) sulfonylureas;
(g) α-glucosidase inhibitors;
(h) agents selected from the group consisting of (i) HMG-CoA reductase inhibitors, (ii) bile acid sequestrants, (iii) nicotinyl alcohol, nicotinic acid or a salt thereof, (iv) niacin receptor agonists, (v) PPARα agonists, (vi) cholesterol absorption inhibitors, (vii) acyl CoA:cholesterol acyltransferase (ACAT) inhibitors, (viii) CETP inhibitors, and (ix) phenolic anti-oxidants;
(i) PPARα/γ dual agonists,
(j) PPARδ agonists,
(k) antiobesity compounds,
(l) ileal bile acid transporter inhibitors;
(m) anti-inflammatory agents;
(n) glucagon receptor antagonists;
(o) GLP-1;
(p) GIP-1; and
(q) GLP-1 analogs; and
(3) a pharmaceutically acceptable carrier.
